(19) United States
(12) Patent Application Publication
(10) Pub. No.: US 2012/0171150 A1
(43) Pub. Date: Jul. 5, 2012

(54) Ion Bonding Pharmaceutical Composition

(75) Inventor: Benjamin S. Metelits, Phoenix, AZ (US)

(73) Assignee: Dr. Benjamin S. Metelits, Phoenix, AZ (US)

(21) Appl. No.: 12/985,023

(22) Filed: Jan. 5, 2011

(51) Int. Cl. A61K 31/795; A61K 31/715; A61K 31/785; C07F 7/02; A61P 9/12; C08F 128/02; A61K 31/695; C07H 15/26; A61K 36/06

(52) U.S. Cl. 424/78.01; 536/55.1; 536/18.7; 536/123; 514/61; 424/78.16; 424/195.16; 525/328.5; 514/63; 540/465; 549/206; 549/212

(57) ABSTRACT

A pharmaceutical composition is provided that is useful for reducing sodium in the bloodstream of animals. In some embodiments, the pharmaceutical composition comprises a high-ion-affinity part and a substrate part. Additionally, methods of using the pharmaceutical composition and methods of treating diseases with the pharmaceutical composition are disclosed.

### BACKGROUND

Hypertension is the most important modifiable risk factor for coronary artery disease, stroke, congestive heart failure, kidney failure, and peripheral vascular disease. Coronary artery disease is the leading cause of death in North America and stroke is the third leading cause of death.

Specifically, hypertension has been linked to myocardial infarction, atrial fibrillation, ventricular arrhythmias including sudden cardiac death, systolic and diastolic heart failure, aortic aneurysms, hemorrhagic stroke, ischemic stroke, dementia, and end-stage renal disease (requiring dialysis or kidney transplant).

Almost all national and international organizations recommend dietary sodium reduction for the treatment of hypertension. Traditionally, drug therapy controls hypertension. Drug therapy can employ multiple classes of anti-hypertensive medications that function through different mechanisms. The most common first-line agents are thiazide diuretics, ACE inhibitors and angiotensin II receptor blockers, and calcium channel blockers. Diuretics, or water pills, are also used and work by promoting the excretion of sodium and water. Decreasing the amount of excess body water reduces the amount of fluid flow in the blood vessels. And decreasing sodium levels decreases blood water volume.

A recent study published in New England Journal of Medicine recommends the reduction of salt by 1-3 g per day. Such a reduction in salt consumption by Americans over the next decade could result in a 10-24 billion dollar savings in health care costs annually. Up until now, sodium reduction in the diet was accomplished by behavioral or lifestyle modification, but has largely been a failure with sodium consumption rising over the last few years.

Almost everyone, even people without hypertension can benefit from low salt consumption. But those most likely to benefit from reduced salt intake are those who already have hypertension. Modifications that decrease blood pressure (e.g., limiting sodium, limiting alcohol, exercising, and losing weight) will decrease the risk of coronary artery disease, stroke, congestive heart failure, kidney failure, and peripheral vascular disease.

For people who have already developed these complications, maintaining low blood pressure is essential to preventing the progression of the disease. And reducing salt consumption works to maintain low blood pressure.

For people with congestive heart failure, maintaining a low-salt diet is crucial. Consuming large amounts of sodium can upset the fluid balance in the body, which can lead to acute decompensated heart failure or a “congestive heart failure exacerbation”. This is a common cause of hospitalization and can be deadly.

Patients with kidney disease or liver disease must also follow a low-salt diet. These two conditions cause retention of water that can lead to lower extremity edema (swelling of the calves), ascites (fluid in the belly), and pulmonary edema (fluid in the lungs). Also, excessive salt consumption can aggravate Meniere's disease.

What is needed is a pharmaceutical that reduces sodium blood levels while being safe for use in humans.

### SUMMARY

The inventor has discovered a novel pharmaceutical composition that is adapted for enteral delivery and that comprises a high-ion-affinity part and a substrate part. The high-ion-affinity part has a high affinity for ions present in the intestinal tract of an animal, such as sodium, potassium, magnesium, or calcium. The high-ion-affinity part, in some embodiments, selectively has a high affinity for sodium or other ions.

In some embodiments the high-ion-affinity part comprises a material comprising cryptands, crown ethers, azacrown ethers, thiacrown ethers, calixarenes, calixcrowns, lariat ethers, calixtubes, crown porphyrins, or diester crown ethers. In some embodiments the high-ion-affinity part is restricted to cryptands or crown ethers.

The pharmaceutical composition additionally comprises a substrate part that may be selected from any number of substrates as are known to those of ordinary skill in the pharmaceutical arts. In some embodiments, suitable substrates are selected from dietary fiber materials, polymer or oligomer materials, ceramic materials, clay materials, or other well-known pharmaceutical substrate materials. Additionally, in some embodiments the substrate part comprises ion exchange materials or potassium-replaced Kayexalate.

The composition can additionally comprise other pharmaceutical drugs. In some embodiments, the other pharmaceutical drugs are selected from hypertension drugs, high-cholesterol-treating drugs, or high-triglyceride-treating drugs.

In some embodiments the additional pharmaceutical or drug is selected from one or more drugs selected from any one or any combination of acebutolol; acetazolamide; aliskiren; amiloride and hydrochlorothiazide; amiloride hydrochloride; amlodipine; amlodipine and benazepril; atenolol; atenolol and chlorthalidone; benazepril; benazepril and hydrochlorothiazide; betaxolol; bisoprolol; bisoprolol and hydrochlorothiazide; bumetanide; candesartan; captopril; captopril and hydrochlorothiazide; carteolol; carvedilol; chlorthalidone; clonidine; diltiazem; doxazosin; enalapril; enalapril and hydrochlorothiazide; esidrix; ethacrynic acid; felodipine; felodipine and enalapril; fosinopril; furosemide; guanabenz; guanfacine; hydralazine; hydralazine and hydrochlorothiazide; hydrochlorothiazide; indapamide; irbesartan; isradipine; labetalol; lisinopril; lisinopril and hydrochlorothiazide; losartan; losartan and hydrochlorothiazide; methyldopa; methyldopa and hydrochlorothiazide; metolazone; metoprolol; metoprolol and hydrochlorothiazide; microzide; moexipril; nadolol; nadolol and bendroflumethiazide; nicardipine; nifedipine; nisoldipine; olmesartan; penbutolol; prazosin; propranolol; propranolol and hydrochlorothiazide; quinapril; ramipril; reserpine; spironolactone; spironolactone and hydrochlorothiazide; telmisartan; terazosin; timolol; torsemide; trandolapril; triamterene; triamterene and hydrochlorothiazide; valsartan; verapamil; verapamil (extended release) and trandolapril; lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

### DETAILED DESCRIPTION

While the discussion below speaks of complexing sodium, other ions that are deleterious to an organism may also be complexed or removed using inventive compounds. Typically, inventive compound can be prepared with different chelating parts—a part that shows a preference for one ion over another. (These are also called high ion affinity parts throughout this disclosure.) By correctly choosing the chelating part, one of ordinary skill in the art can choose which ions are bound and eliminated by the digestive process versus which ions remain in solution and eventually migrate into the blood stream of the organism. In various embodiments, the ion selected for removal is sodium, potassium, magnesium, or calcium. In these or other embodiments, urea or uric acid is removed by inventive compounds.

Sodium is contained in all of the food an organism consumes. Additionally, sodium chloride is added (or over added) to season food. Inventive compounds are suitable for delivery to the intestinal tract of an organism and, once there, binding with the free sodium ions in solution in the intestine. Binding to the compounds removes the sodium ions from solution in the digestive fluid lowering the sodium concentration in the digestive fluid because the degree to which sodium passes from solution in the digestive fluid across the blood barrier or into the bloodstream is proportional to the digestive-side concentration. Therefore, pulling sodium out of solution on the intestine side of the blood barrier equates to less sodium entering the bloodstream of the organism.

The normal digestive process eliminates the inventive compound along with the entrained or complexed sodium.

As will be discussed below, the inventive compounds contain a chelating part that interacts with the sodium ion at least one portion of the chelating part. In some embodiments, this interaction is with two or more portions of the chelating part calling into play the chelate effect, which statistically heightens the binding effect of the inventive compound on the sodium ion.

Also, the typical interaction between an ion and the inventive compound is electrostatic. But any interaction between the inventive compound and sodium can function to lower serum sodium levels in the organism and falls within the scope of this disclosure.

### Chelating or High Ion Affinity Portion or Part

The main features of a high affinity high ion affinity portion of an inventive compound according to some embodiments of the invention include the following: the ability to complex a target ion; the ability to complex a target ion somewhat selectively over other non-target ions; the ability to couple with a substrate; substantial non-toxicity, either inherently or when coupled to the substrate; or substantial stability in the intestinal fluid.

In some embodiments, the high ion affinity portion has a high affinity for sodium ions. In these or other embodiments, the high ion affinity portion has a higher affinity for sodium ions than for potassium ions, calcium ions, or magnesium ions.

Crown ethers, a type of chelating agent, show a strong affinity for alkali and alkaline earth ions. These molecules are macrocyclic polyethers that can contain 3-20 oxygen atoms. Within the macrocyclic ring, two or more carbon atoms are spaced apart by oxygen atoms. In the simpler crown ether examples, the geometry of the macrocyclic ring coupled with the angle between carbon atoms enforced by the oxygen atom geometry gives a crown-like structure with the oxygen atoms sitting at the points of the crown—hence the name crown ether. This arrangement forces the non-bonding electron pairs on the oxygen atoms to create a cavity surrounded by electron density—a region with a high affinity for a number of metal cations.

The common nomenclature of the crown ethers contains the number of atoms in the macrocyclic ring and the number of oxygen atoms in the ring. Hence 15-crown-5 contains 15 atoms in the macrocyclic ring, five of which are oxygen atoms, see Structure I. Another example is dibenzo-18-crown-6, see Structure II.

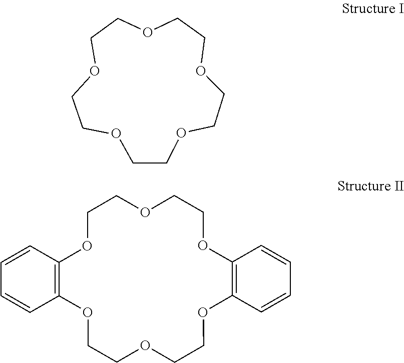

Additionally, other heteroatoms such as nitrogen, phosphorus, and sulfur can replace the macrocyclic oxygen atoms giving rise to azacrown ethers, phosphacrown ethers, and thiacrown ethers. For example, aza-12-crown-4 is shown in Structure III.

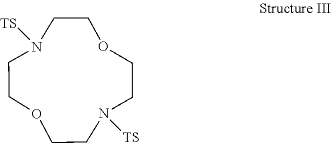

Substitution of at least some of the oxygen atoms by different heteroatoms such as nitrogen provides a coordination site for attaching additional carbon-containing groups to add functionality to the molecule. For example, one or more nitrogen atoms can accept a side group or side arm. Provisioning the side arm with its own set of complexing parts or chelating parts such as ethoxy-based parts creates prototypical lariat ethers, as shown in Structure IV.

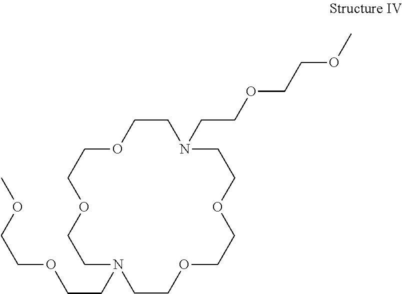

Alternatively, one or more nitrogen atoms can instead be bridged. Provisioning the bridge with complexing atoms creates a three-dimensional structure with a coordination site for the cations inside of the structure. Structure V shows this prototypical cryptand.

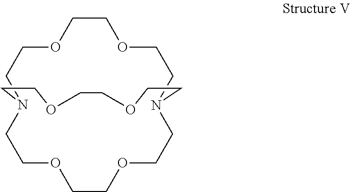

Another type of cation binding molecule is based on calixarene. Calixarenes are macrocycles formed by cyclo-polymerization of phenol and formaldehyde. In addition to their innate ability to complex cations, calixarenes have been hybridized into crown ethers making calixcrowns. Similar forms with nitrogen and sulfur substitution are known, as well, with 3-D structures reminiscent of cryptands formed by bridging calixarenes.

### Substrates

A variety of different materials are suitable for use as substrates in invention compositions: soluble dietary fiber; insoluble dietary fiber; pharmaceutical grade polymer; ion exchange resins, such as Kayexalate in which sodium has been substantially replaced by potassium; kaolin; pectin; clay; starch; metal oxides; and other pharmaceutical substrates. Several features useful for a substrate according to the compositions of the invention include the following: substantial stability in the intestinal fluid; substantial lack of toxicity; or the ability to be coupled with a high ion affinity part.

In some embodiments, the substrate is capable of being chemically coupled to an high ion affinity part. Additionally, a useful substrate will function to cause the high ion affinity part to remain in the intestinal tract and not cross the blood barrier into the bloodstream.

### Fiber Substrate

A suitable dietary fiber substrate for use in invention compositions will meet the functional definition stated above and have a soluble or insoluble fiber nature or a resistant starch nature. In some embodiments, the substrate is selected from one of the following materials or mixtures of these materials: glycosaminoglycans; homogalacturonans; substituted galacturonans; rhamnogalacturonans; hyaluronic acid; carboxymethyl cellulose; pectin; hydroxypropyl methylcellulose; hydroxypropyl cellulose; methylcellulose; sodium carboxymethylcellulose; hydroxyethylcellulose; acacia; tragacanth; guar gum; xanthan gum; locust bean gum; polysaccharides; natural gum; agar; agarose; sodium alginate; carrageenan; fucoidan; furcellaran; laminaran; hypnea; eucheuma; gum arabic; gum ghatti; gum karaya; arbinoglactan; amylopectin; gelatin; carboxylmethyl cellulose gum; alginate gum; psyllium fiber; bran fiber; wheat bran fiber; oat fiber; and wheat fiber.

### Dietary Fiber

Most of these fiber compounds are polysaccharides or oligosaccharides. These are essentially polymers of glucose molecules. Thus, these chains have accessible hydroxyl groups along their length.

### Soluble Fiber

According to the American Association of Cereal Chemists, soluble fiber is “the edible parts of plants or similar carbohydrates resistant to digestion and absorption in the human small intestine with complete or partial fermentation in the large intestine”.

### Insoluble Fiber

Insoluble fiber is the indigestible portion of plant food that is metabolically inert, but water absorbing throughout the digestive tract. Metabolically inert means that the chemical structure of the fiber is not substantially changed during its trip through the digestive tract.

### Resistant Starch

Resistant starches are starches and their degradation products that are not substantially digested in the small intestine.

### Polymers

A large variety of polymers are safe for delivery to the intestinal tract of humans. A suitable polymer substrate for use in invention compositions will meet the functional definition stated above and additionally be polymeric or copolymeric in nature. For purposes of this disclosure, polymers include oligomers composed of three or more monomer repeat units. In some embodiments, the substrate is selected from one of the following materials or mixtures of these materials: carboxyvinyl polymers; methacrylic acid-based polymers; polyorthocarbonates; 2-methacryloyloxyethylphosphorylcholine (MPC); 3-trimethylsilylpropyl methacrylate (TMSPMA); ABS resins; acrylic acid polymers; acrylic acid-based polymers; acrylic copolymers; acrylonitrile-styrene copolymers; albumen; alginates; alkoxyacrylate; alkoxymethacrylate; alkyd resins; ammonium methacrylate copolymers; carboxylic acid bearing monomers; cellophane; cellulose acetate; cellulose acetate butyrate; cellulose acetate phthalate; cellulose butyrate; cellulose ethers; cellulose nitrate; cellulose propionate; cellulosic polymers; collagen; COPA; dendrimers; dextrins; epoxy resins; ePTFE; ETFE (ethylene and tetrafluoroethylene co-polymer); ethylcellulose; ethylene vinyl acetate; ethylene-methyl methacrylate copolymers; ethylene-vinyl acetate copolymers; fluorinated ethylene propylene; fluoropolymers; GAG derivatives; gelan gum; gelatin; gellan gum; glycosamino glycan (GAG); gum acacia; gum arabic; HEMA; heteropolysaccharide polymer; hyaluronic acid; hydroxy alkyl celluloses; hydroxy cellulose; hydroxy functional poly(vinyl pyrrolidone); hydroxyethyl cellulose; hydroxyethyl methylcellulose; hydroxypropyl methacrylate (HPMA); hydroxypropyl methyl cellulose (HPMC); hydroxypropyl methyl cellulose phthalate; hydroxypropylcellulose; hydroxypropylmethacrylamide; methacrylic acid; methacrylic acid copolymers; methyl cellulose; N-vinyl pyrrolidone; nylon; Nylon 66; PAMAM starburst dendrimer; Parylast; Parylene; PCTFE (polychlorotrifluoroethylene); PEBAX; PEG acrylate (PEGA); PEG and PEO mixtures; PEG methacrylate; PFA (perfluoroalkyl vinyl ether and tetrafluoroethylene co-polymer); phosphoryl choline; PLA-PEG; PLURONIC™ surfactants (polypropylene oxide-co-polyethylene glycol); poly(3-hydroxyalkanoates); poly(4-hydroxyalknaotes); poly(alpha-aminoacids); poly(alpha-aminoacids) copolymers; poly(alpha-hydroxy acids); poly(alpha-hydroxy acids) copolymers; poly(amino acids); poly(anhydrides); poly(aspirin); poly(caprolactone); poly(D,L-lactide); poly(D,L-lactide-co-glycolide); poly(dioxanone); poly(ester amide); poly(ether-esters); poly(ethyl methacrylate); poly(ethylene oxide-co-lactic acid) (PEO/PLA); poly(glyceryl sebacate); poly(glycolic acid-co-trimethylene carbonate); poly(glycolide-co-caprolactone); poly(imino carbonates); poly(iminocarbonate); poly(isobutyl methacrylate); poly(isopropyl methacrylate); poly(lactide-co-caprolactone); poly(lactide-co-glycolide); poly(L-lactide); poly(L-lactide-co-glycolide); poly(methyl methacrylate); poly(methyl methacrylate)-PEG (PMMA-PEG); poly(n-butyl methacrylate); poly(n-propyl methacrylate); poly(ortho esters); poly(phosphoesters); poly(propylene fumarate); poly(propylene oxide); poly(sec-butyl methacrylate); poly(styrene-isoprene-styrene)-PEG (SIS-PEG); poly(tert-butyl methacrylate); poly(tetramethylene glycol); poly(trimethylene carbonate); poly(tyrosine carbonates); poly(tyrosine ester) derivatives; poly(vinylidene fluoride-co-hexafluoropropylene); polyacrilamide; polyacrylates; polyacrylic acid; polyacrylonitrile; polyalkylene glycols; polyalkylene oxalates; polyalkylene oxides; polyamides; polyamidoamine; polyamino acids; polyanhydrides; polycaprolactam; polycaprolactone; polycaprolactone-PEG (PCL-PEG); polycarbonates; polycyanoacrylates; polydimethylsiloxane-co-PEG (PDMS-PEG); polyesters; polyethers; polyethylene; polyethylene glycols (PEG); polyethylene oxides (PEO); polyethylene terephthalate; polyglycolide; polyglycolysed glycerides; polyhydroxyalkanoates (PHA); polyimides; polyisobutylene and ethylene-alphaolefin copolymers; polyisobutylene-PEG; polymers and copolymers comprising a monomer selected from the group consisting of 3-hydroxypropanoate, 3-hydroxybutyrate, 3-hydroxyvalerate, 3-hydroxyhexanoate, 3-hydroxyheptanoate, 3-hydroxyoctanoate, 4-hydroxybutyrate, 4-hydroxyvalerate, 4-hydroxyhexanote, 4-hydroxyheptanoate, or 4-hydroxyoctanoate; polymers and co-polymers of hydroxyl bearing monomers; polymethacrylate copolymers; polymethacrylates; polyolefins; polyorthoesters; polyoxamers; polyoxyethylene-polyoxypropylene copolymers; polyoxymethylenes; polyphosphazenes; polyphosphoester; polyphosphoester urethane; polypropalkylenes; polypropylenes; polystyrene homo- and copolymers; polystyrene-PEG; polystyrenes; polyurethanes; polyvinyl acetates; polyvinyl alcohol (PVA); polyvinyl aromatics; polyvinyl chloride; polyvinyl esters; polyvinyl ethers; polyvinyl ketones; polyvinyl methyl ether; polyvinylidene chloride; polyvinylidene halides; polyvinylpyrollidones; PTFE; pullulan; PVDF (polyvinylidenedifluoride); PVF (polyvinylfluoride); rayons; rayon-triacetate; shellac; silicones; TFB (terpolymer of vinylidenedifluoride, hexafluoropropylene and tetrafluoroethylene); vinyl halide copolymers; vinyl halide polymers; vinyl monomer olefin copolymers; vinyl monomers copolymers; xanthan gum; and mixtures and copolymers of these polymers.

### Clay Materials

A large variety of clays are safe for delivery to the intestinal tract of humans. A suitable clay substrate for use in invention compositions will meet the functional definition stated above and additionally comprise a clay. In some embodiments, the substrate is selected from one of the following materials or mixtures of these materials: kaolin or kaolinite; smectite clays; illite clays; bentonite; montmorillonite; Fuller's earth; calcium bentonite; palygorskite; or attapulgite.

### Ceramic Materials

A variety of ceramic materials, perhaps all, are safe for delivery to the intentinal tract of humans. Suitable ceramics are substantially refractory materials that are substantially inert to the intenstinal environment and mee the substrate functional definitions stated above. In some embodiments, the substrate is selected from one of the following materials or mixtures of these materials: silica; alumina; hydroxyapatite; alumina; zirconia; titania; yttria; silicon carbide; silicon nitride; aluminum carbide; aluminum nitride; and tungsten carbide.

### Miscellaneous Substrate Materials

For purposes of this disclosure, miscellaneous substrate materials are defined to include the following materials and materials functionally similar to these materials vis-à-vis the disclosed invention: diatomaceous earths; zeolites; graphite; transition metal oxides; and others. Diatomaceous earth also known as diatomite or kieselgur, is a naturally occurring, soft, siliceous sedimentary rock. It consists of the fossilized remains of diatoms. Zeolites are microporous, aluminosilicate minerals. Transition metal oxides are compounds containing one or more metals atoms from the transition metal series of the Periodic Table combined with at least some oxygen atoms.

### Synthesis

The compositions of the invention comprise a high ion affinity part coupled to a substrate, as each were described above. The coupling between the components can be carried out through any structure or methodology as is known to skilled artisans. As those of ordinary skill in the art are well versed in a myriad of ways of carrying out this type of coupling, the following synthetic regimes are exemplary only and not intended to limit the scope of the attached claims.

Features of the coupling part include the ability to create a robust physical connection between a high ion affinity part and the substrate. In some embodiments, the coupling part is robust enough to withstand the environment in the intestinal tract. Also, the coupling part in some embodiments is selected such that it does not interfere with the function of the substrate or the function of the high ion affinity part more than necessary or more than one of ordinary skill in the art would deem acceptable. In some embodiments, the coupling part adds no toxicity to the composition. In other embodiments, the toxicity added by the coupling is of a degree that one of ordinary skill in the art would deem acceptable for pharmaceutical compositions.

In some embodiments, the compositions are specifically adapted for delivery as a pharmaceutical. Such adaptations may include any adaption recognized by one of ordinary skill in the art to make the compounds more suitable for delivery as a pharmaceutical.

In some embodiments, the compositions are specifically adapted for enteral delivery includes any of a variety of adaptations as are recognized by those of ordinary skill in the art to make a pharmaceutical composition more suitable for controlled delivery. These include, but are not limited to, making the composition easy to swallow such as by forming it into tablets or pills and optionally coating the tablets or pills. Alternatively, the composition may be formulated as a liquid medication. The composition may contain features that cause its components to withstand the digestive environment that enteral delivery exposes it to. Alternatively or in conjunction with that, the composition may be micro-encapsulated such that it does not become active in the digestive tract until it reaches the higher pH regions of the digestive tract or until it reaches the small intestine.

Adapted for enteral delivery may also include modifications to the composition or selection of components of the composition that will lower toxicity.

### Polyurea or Polyurethane Linkages with Dietary Fiber

The polysaccharide structure of dietary fiber and related substrates comprises a chain of glucose molecules, which have accessible hydroxyl groups. The chemistry to amidate the fiber changes some of these accessible hydroxyl groups into amino groups and is well known and widely practiced. Functionalizing a high ion affinity part with an organic group comprising accessible or terminal isocyanate group is also well-known and straightforward chemistry. See Structure VI for a depiction of an isocyanate-substited cryptand.

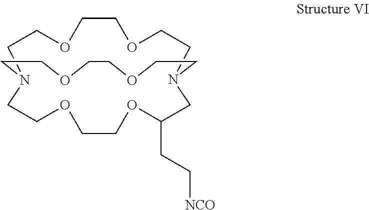

Reaction of the polysaccharide molecules with the isocyanate-functionalized high ion affinity part will yield a urethane coupling between the high ion affinity part and the polysaccharide substrate, as shown in Structure VIII. The reaction of the amino functionalized polysaccharide with the isocyanate-functionalized high ion affinity part will yield a urea coupling between the high ion affinity part and the polysaccharide substrate, as shown in Structure VIII.

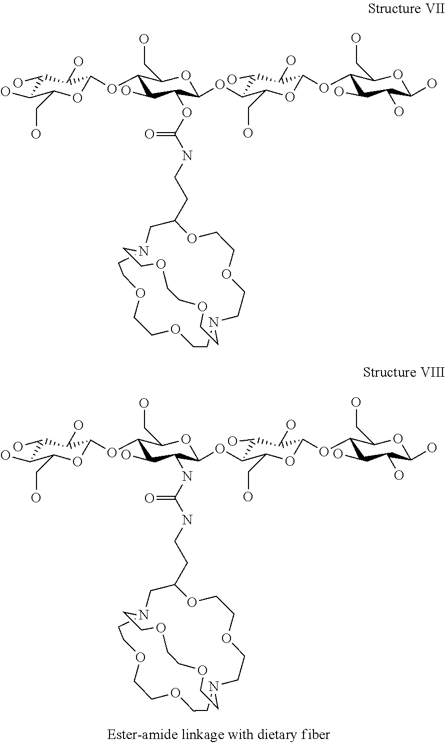

Alternatively, the coupling part may comprise a carboxyl group and thus when added to the high ion affinity part will form a carboxyl-functionalized high ion affinity part. A high ion affinity part of 15-crown-5 with a carboxyl-coupling group is shown in Structure IX.

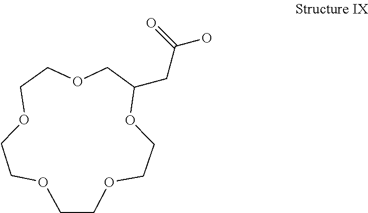

When the carboxyl-functionalized high ion affinity part is reacted with the polysaccharide, the resulting inventive compound comprises an ester coupling between the high ion affinity part and the polysaccharide substrate as shown in Structure X.

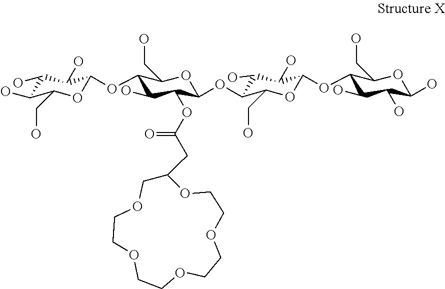

Similarly when the carboxylate-functionalized high ion affinity part is reacted with the amidated polysaccharide, the resulting inventive compound comprises an amide coupling between the high ion affinity part and the polysaccharide substrate, as shown in Structure XI.

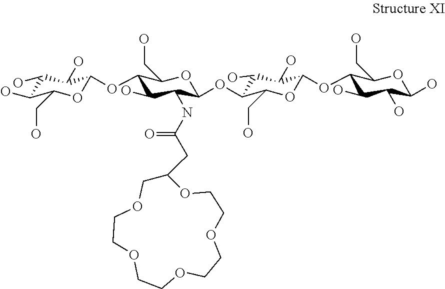

Another method of creating an amide linkage or an ether linkage between the high ion affinity part and the polysaccharide substrate is to react the polysaccharide substrate to carboxylate it. This chemistry is well known. When the carboxylated fiber is added to a hydroxyl functionalized high ion affinity part or a high ion affinity part comprising an organic moiety containing a hydroxyl part and ether linkage is formed between the high ion affinity part and the polysaccharide substrate. Likewise, if the carboxylated fiber is added to an amino functionalized high ion affinity part or a high ion affinity part comprising an organic group containing an amino group or an amine, the resulting compound comprises an amide linkage between the polysaccharide substrate and the high ion affinity part.

### PEG-Based Coupling with Dietary Fiber

Either the high ion affinity part or substrate can be functionalized with a short-chained polyethylene glycol. The dehydration reaction between the peg-functionalized moiety and a hydroxyl group on the other component will yield a peg-coupled pharmaceutical composition of the invention. This composition will comprise a high ion affinity part coupled to a short-chained peg part that in turn couples to the substrate. This can be seen in Structure XII.

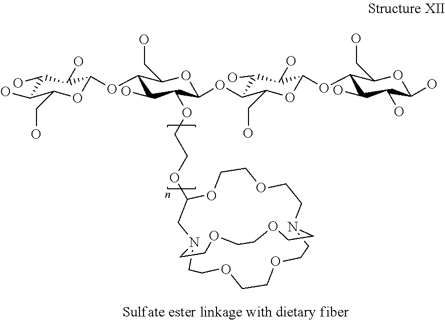

The fiber or other substrate can be reacted to yield sulfate group functionalities. One way of doing this is disclosed in U.S. Pat. No. 4,141,746, which is incorporated herein in its entirety. The high ion affinity part has a hydroxyl group or can be functionalized to have a hydroxyl group. When the sulfated fiber and the alcohol group in a high ion affinity part react, they yield sulfate ester coupling between the high ion affinity part and the substrate. See Structure XIII. Alternatively, a sulfonic acid group can be added to the fiber, which when reacted with the high ion affinity part, yields a sulfonic acid ester.

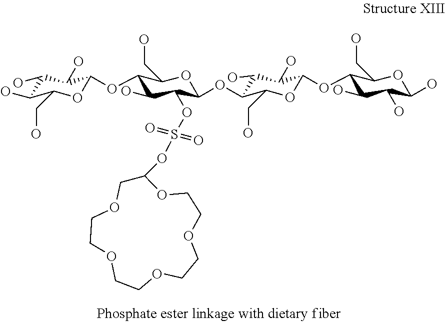

The fiber or other substrate can be reacted to yield phosphate group functionalities. The high ion affinity part has a hydroxyl group or can be functionalized to have a hydroxyl group. When the phosphate fiber and the alcohol group in a high ion affinity part react, they yield phosphate ester coupling between the high ion affinity part and the substrate.

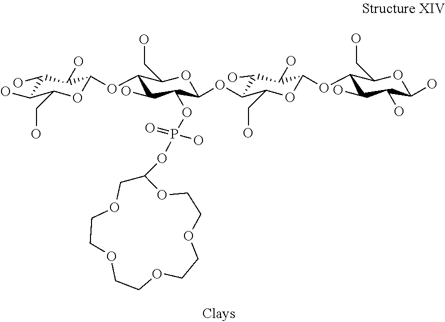

As with fiber-based substrates, clay-based substrates can be reacted to contain a sulfate group or the clays can natively contain a sulfate group. When the sulfate group is reacted with an alcoholic functionality added to the high ion affinity part, a sulfate ester coupling the high ion affinity part to the clay substrate is created.

As with fiber-based substrates, clay-based substrates can be reacted to contain a phosphate group or the clays can natively contain a phosphate group. When the phosphate group is reacted with an alcoholic functionality added to the high ion affinity part, a phosphate ester coupling the high ion affinity part to the clay substrate is created.

### Polyurea or Polyurethane Linkages and Polymer Substrate

Kayexalate in which sodium has been substantially replaced by potassium is widely used in treating potassium ion overabundances. It comprises a polystyrene sulfonate polymer with sodium ions. Polystyrene sulfonate polymers are typical ion exchange resins meaning that they are capable of complexing or electrostatically interacting with ions. The potassium analog will exchange sodium ions in solution thereby reducing the amount of sodium ions in solution.

The polymer structure of polystyrene and related substrates comprises a polyethylene chain substituted with a benzene sulfonate group once per repeat unit. The chemistry to add hydroxyl or amine groups to the polymer is well known. See Structure VI for a depiction of an isocyanate-substituted cryptand.

Reaction of the hydroxylated polymer molecules with the isocyanate-functionalized high ion affinity part will yield a urethane coupling between the high ion affinity part and the polymer substrate, as shown in Structure XV. The reaction of the amino functionalized polymer with the isocyanate-functionalized high ion affinity part will yield a urea coupling between the high ion affinity part and the polymer substrate, as shown in Structure XVI.

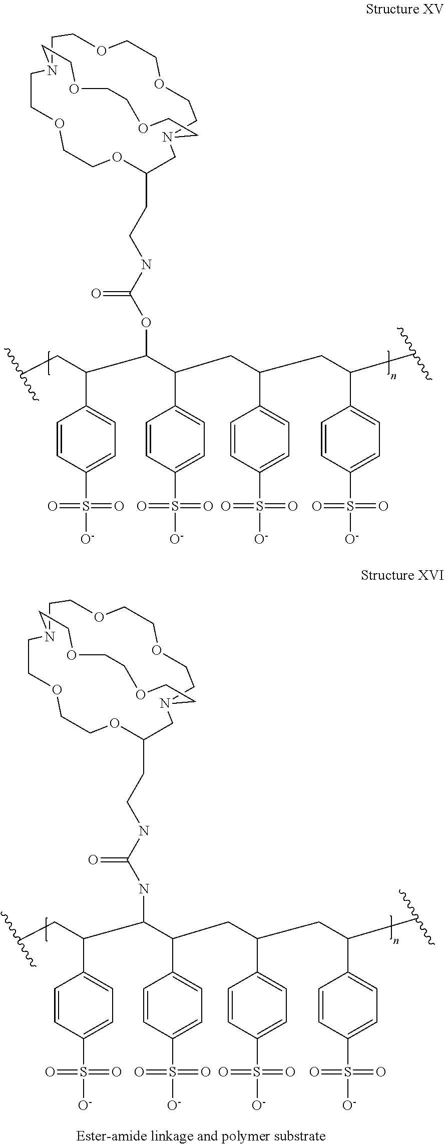

When the carboxyl-functionalized high ion affinity part is reacted with the polymer, the resulting inventive compound comprises an ester coupling between the high ion affinity part and the polymer substrate as shown in Structure XVII.

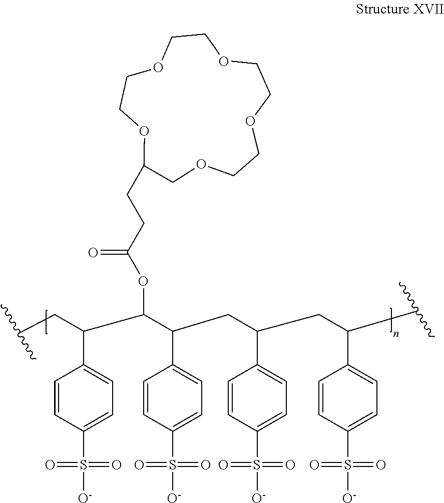

Similarly, when the carboxylate-functionalized high ion affinity part is reacted with the amidated polymer, the resulting inventive compound comprises an amide coupling between the high ion affinity part and the polymer substrate.

Another method of creating an amide linkage or an ether linkage between the high ion affinity part and the polymer substrate is to react the polymer substrate to carboxylate it. This chemistry is well known. When the carboxylated polymer is added to a hydroxyl functionalized high ion affinity part or a high ion affinity part comprising an organic moiety containing a hydroxyl part and ester linkage is formed between the high ion affinity part and the polymer substrate. Likewise, if the carboxylated polymer is added to an amino functionalized high ion affinity part or a high ion affinity part comprising an organic group containing an amino group or an amine, the resulting compound comprises an amide linkage between the polymer substrate and the high ion affinity part.

### PEG-Based Coupling with Polymer Substrate

Either the high ion affinity part or substrate can be functionalized with the short-chained polyethylene glycol. The dehydration reaction between the peg-functionalized moiety and a hydroxyl group on the other component will yield a peg-coupled pharmaceutical composition of the invention. This composition will comprise a high ion affinity part coupled to a short-chained peg part that in turn couples to the substrate.

### Dosage Considerations

The amount of sodium removed from the intestinal tract of a treated individual depends on a number of factors including the amount of sodium present in the intestines and the amount of high ion affinity component coupled to the fiber or the substrate. Higher concentrations of sodium ions would yield a greater number of complexed ions in the high ion affinity portion based solely on equilibrium considerations.

With regard to high ion affinity part content, 5 g of a composition prepared with one [2.2.2]-crypt on each glucose unit of a polysaccharide, such as dietary fiber, has the capacity to complex 230 mg of sodium ions.

5 g of the material made of potassium polystyrene sulfonate (Kayexalate in which sodium has been substantially replaced by potassium ion-exchanged with potassium ions) as the substrate with [2.2.2]-crypt coupled along the substrate backbone, one cryptand per three repeating units of the backbone would have a capacity for 120 mg of sodium taken up by the cryptand and 360 mg of sodium taken up by the ion exchange of sodium for potassium in the substrate.

5 g of material prepared with one molecule of 15-crown-5 on each glucose unit of a polysaccharide dietary fiber chain has a capacity to complex 294 mg of sodium.

### Other Drugs

The compositions of the invention can be combined with any drug or combination of drugs useful for treating diseases in humans. For instance, the compositions of the invention are suited for combining with one or more hypertension drugs, one or more high-cholesterol- or -triglyceride-treating drugs, or any combination of hypertension drugs and high-cholesterol- or -triglyceride-treating drugs.

### Hypertension Drugs

The following classes of drugs are used to treat hypertension: angiotensin-converting enzyme (ace) inhibitors; angiotensin ii receptor blockers; beta blockers; calcium channel blockers; renin inhibitors; and thiazide diuretics.

The following drugs are used to treat hypertension: acebutolol; acetazolamide; aliskiren; amiloride and hydrochlorothiazide; amiloride hydrochloride; amlodipine; amlodipine and benazepril; atenolol; atenolol and chlorthalidone; benazepril; benazepril and hydrochlorothiazide; betaxolol; bisoprolol; bisoprolol and hydrochlorothiazide; bumetanide; candesartan; captopril; captopril and hydrochlorothiazide; carteolol; carvedilol; chlorthalidone; clonidine; diltiazem; doxazosin; enalapril; enalapril and hydrochlorothiazide; esidrix; ethacrynic acid; felodipine; felodipine and enalapril; fosinopril; furosemide; guanabenz; guanfacine; hydralazine; hydralazine and hydrochlorothiazide; hydrochlorothiazide; indapamide; irbesartan; isradipine; labetalol; lisinopril; lisinopril and hydrochlorothiazide; losartan; losartan and hydrochlorothiazide; methyldopa; methyldopa and hydrochlorothiazide; metolazone; metoprolol; metoprolol and hydrochlorothiazide; microzide; moexipril; nadolol; nadolol and bendroflumethiazide; nicardipine; nifedipine; nisoldipine; olmesartan; penbutolol; prazosin; propranolol; propranolol and hydrochlorothiazide; quinapril; ramipril; reserpine; spironolactone; spironolactone and hydrochlorothiazide; telmisartan; terazosin; timolol; torsemide; trandolapril; triamterene; triamterene and hydrochlorothiazide; valsartan; verapamil; and verapamil (extended release) and trandolapril.

### High-Cholesterol- or -Triglyceride-Treating Drugs

The following classes of drugs are used to treat high cholesterol or high triglycerides: lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; and pitavastatin.

### Miscellaneous Embodiments

A composition comprising a high-ion-affinity part and a substrate part wherein the composition is adapted for enteral delivery and wherein the high-ion-affinity part comprises a material comprising cryptands.

A composition comprising a high-ion-affinity part and a substrate part wherein the composition is adapted for enteral delivery and wherein the high-ion-affinity part comprises a material comprising crown ethers.

A composition comprising a high-ion-affinity part and a substrate part wherein the composition is adapted for enteral delivery and wherein the high-ion-affinity part comprises a material comprising azacrown ethers.

A composition comprising a high-ion-affinity part and a substrate part wherein the composition is adapted for enteral delivery and wherein the high-ion-affinity part comprises a material comprising thiacrown ethers.

A composition comprising a high-ion-affinity part and a substrate part wherein the composition is adapted for enteral delivery and wherein the high-ion-affinity part comprises a material comprising calixarenes.

A composition comprising a high-ion-affinity part and a substrate part wherein the composition is adapted for enteral delivery and wherein the high-ion-affinity part comprises a material comprising calixcrowns.

A composition comprising a high-ion-affinity part and a substrate part wherein the composition is adapted for enteral delivery and wherein the high-ion-affinity part comprises a material comprising lariat ethers.

A composition comprising a high-ion-affinity part and a substrate part wherein the composition is adapted for enteral delivery and wherein the high-ion-affinity part comprises a material comprising calix tubes.

A composition comprising a high-ion-affinity part and a substrate part wherein the composition is adapted for enteral delivery and wherein the high-ion-affinity part comprises a material comprising crown porphyrins.

A composition comprising a high-ion-affinity part and a substrate part wherein the composition is adapted for enteral delivery and wherein the high-ion-affinity part comprises a material comprising diestercrown ethers.

A composition comprising a high-ion-affinity part and a substrate part wherein the composition is adapted for enteral delivery and wherein the high-ion-affinity part comprises a material comprising cryptands and wherein the high-ion-affinity part has a high affinity for sodium.

A composition comprising a high-ion-affinity part and a substrate part wherein the composition is adapted for enteral delivery and wherein the high-ion-affinity part comprises a material comprising crown ethers and wherein the high-ion-affinity part has a high affinity for sodium.

A composition comprising a high-ion-affinity part and a substrate part wherein the composition is adapted for enteral delivery and wherein the high-ion-affinity part comprises a material comprising azacrown ethers and wherein the high-ion-affinity part has a high affinity for sodium.

A composition comprising a high-ion-affinity part and a substrate part wherein the composition is adapted for enteral delivery and wherein the high-ion-affinity part comprises a material comprising thiacrown ethers and wherein the high-ion-affinity part has a high affinity for sodium.

A composition comprising a high-ion-affinity part and a substrate part wherein the composition is adapted for enteral delivery and wherein the high-ion-affinity part comprises a material comprising calixarenes and wherein the high-ion-affinity part has a high affinity for sodium.

A composition comprising a high-ion-affinity part and a substrate part wherein the composition is adapted for enteral delivery and wherein the high-ion-affinity part comprises a material comprising calixcrowns and wherein the high-ion-affinity part has a high affinity for sodium.

A composition comprising a high-ion-affinity part and a substrate part wherein the composition is adapted for enteral delivery and wherein the high-ion-affinity part comprises a material comprising lariat ethers and wherein the high-ion-affinity part has a high affinity for sodium.

A composition comprising a high-ion-affinity part and a substrate part wherein the composition is adapted for enteral delivery and wherein the high-ion-affinity part comprises a material comprising calix tubes and wherein the high-ion-affinity part has a high affinity for sodium.

A composition comprising a high-ion-affinity part and a substrate part wherein the composition is adapted for enteral delivery and wherein the high-ion-affinity part comprises a material comprising crown porphyrins and wherein the high-ion-affinity part has a high affinity for sodium.

A composition comprising a high-ion-affinity part and a substrate part wherein the composition is adapted for enteral delivery and wherein the high-ion-affinity part comprises a material comprising diestercrown ethers and wherein the high-ion-affinity part has a high affinity for sodium.

A composition comprising a high-ion-affinity part and a substrate part wherein the composition is adapted for enteral delivery and wherein the high-ion-affinity part comprises a material comprising cryptands and wherein the substrate part comprises a material comprising dietary fiber materials, polymer materials, ceramic materials, clay materials, or miscellaneous substrate materials.

A composition comprising a high-ion-affinity part and a substrate part wherein the composition is adapted for enteral delivery and wherein the high-ion-affinity part comprises a material comprising crown ethers and wherein the substrate part comprises a material comprising dietary fiber materials, polymer materials, ceramic materials, clay materials, or miscellaneous substrate materials.

A composition comprising a high-ion-affinity part and a substrate part wherein the composition is adapted for enteral delivery and wherein the high-ion-affinity part comprises a material comprising azacrown ethers and wherein the substrate part comprises a material comprising dietary fiber materials, polymer materials, ceramic materials, clay materials, or miscellaneous substrate materials.

A composition comprising a high-ion-affinity part and a substrate part wherein the composition is adapted for enteral delivery and wherein the high-ion-affinity part comprises a material comprising thiacrown ethers and wherein the substrate part comprises a material comprising dietary fiber materials, polymer materials, ceramic materials, clay materials, or miscellaneous substrate materials.

A composition comprising a high-ion-affinity part and a substrate part wherein the composition is adapted for enteral delivery and wherein the high-ion-affinity part comprises a material comprising calixarenes and wherein the substrate part comprises a material comprising dietary fiber materials, polymer materials, ceramic materials, clay materials, or miscellaneous substrate materials.

A composition comprising a high-ion-affinity part and a substrate part wherein the composition is adapted for enteral delivery and wherein the high-ion-affinity part comprises a material comprising calixcrowns and wherein the substrate part comprises a material comprising dietary fiber materials, polymer materials, ceramic materials, clay materials, or miscellaneous substrate materials.

A composition comprising a high-ion-affinity part and a substrate part wherein the composition is adapted for enteral delivery and wherein the high-ion-affinity part comprises a material comprising lariat ethers and wherein the substrate part comprises a material comprising dietary fiber materials, polymer materials, ceramic materials, clay materials, or miscellaneous substrate materials.

A composition comprising a high-ion-affinity part and a substrate part wherein the composition is adapted for enteral delivery and wherein the high-ion-affinity part comprises a material comprising calix tubes and wherein the substrate part comprises a material comprising dietary fiber materials, polymer materials, ceramic materials, clay materials, or miscellaneous substrate materials.

A composition comprising a high-ion-affinity part and a substrate part wherein the composition is adapted for enteral delivery and wherein the high-ion-affinity part comprises a material comprising crown porphyrins and wherein the substrate part comprises a material comprising dietary fiber materials, polymer materials, ceramic materials, clay materials, or miscellaneous substrate materials.

A composition comprising a high-ion-affinity part and a substrate part wherein the composition is adapted for enteral delivery and wherein the high-ion-affinity part comprises a material comprising diestercrown ethers and wherein the substrate part comprises a material comprising dietary fiber materials, polymer materials, ceramic materials, clay materials, or miscellaneous substrate materials.

A composition comprising a high-ion-affinity part and a substrate part wherein the composition is adapted for enteral delivery and wherein the high-ion-affinity part comprises a material comprising cryptands and wherein the high-ion-affinity part has a high affinity for sodium and wherein the substrate part comprises a material comprising dietary fiber materials, polymer materials, ceramic materials, clay materials, or miscellaneous substrate materials.

A composition comprising a high-ion-affinity part and a substrate part wherein the composition is adapted for enteral delivery and wherein the high-ion-affinity part comprises a material comprising crown ethers and wherein the high-ion-affinity part has a high affinity for sodium and wherein the substrate part comprises a material comprising dietary fiber materials, polymer materials, ceramic materials, clay materials, or miscellaneous substrate materials.

A composition comprising a high-ion-affinity part and a substrate part wherein the composition is adapted for enteral delivery and wherein the high-ion-affinity part comprises a material comprising azacrown ethers and wherein the high-ion-affinity part has a high affinity for sodium and wherein the substrate part comprises a material comprising dietary fiber materials, polymer materials, ceramic materials, clay materials, or miscellaneous substrate materials.

A composition comprising a high-ion-affinity part and a substrate part wherein the composition is adapted for enteral delivery and wherein the high-ion-affinity part comprises a material comprising thiacrown ethers and wherein the high-ion-affinity part has a high affinity for sodium and wherein the substrate part comprises a material comprising dietary fiber materials, polymer materials, ceramic materials, clay materials, or miscellaneous substrate materials.

A composition comprising a high-ion-affinity part and a substrate part wherein the composition is adapted for enteral delivery and wherein the high-ion-affinity part comprises a material comprising calixarenes and wherein the high-ion-affinity part has a high affinity for sodium and wherein the substrate part comprises a material comprising dietary fiber materials, polymer materials, ceramic materials, clay materials, or miscellaneous substrate materials.

A composition comprising a high-ion-affinity part and a substrate part wherein the composition is adapted for enteral delivery and wherein the high-ion-affinity part comprises a material comprising calixcrowns and wherein the high-ion-affinity part has a high affinity for sodium and wherein the substrate part comprises a material comprising dietary fiber materials, polymer materials, ceramic materials, clay materials, or miscellaneous substrate materials.

A composition comprising a high-ion-affinity part and a substrate part wherein the composition is adapted for enteral delivery and wherein the high-ion-affinity part comprises a material comprising lariat ethers and wherein the high-ion-affinity part has a high affinity for sodium and wherein the substrate part comprises a material comprising dietary fiber materials, polymer materials, ceramic materials, clay materials, or miscellaneous substrate materials.

A composition comprising a high-ion-affinity part and a substrate part wherein the composition is adapted for enteral delivery and wherein the high-ion-affinity part comprises a material comprising calix tubes and wherein the high-ion-affinity part has a high affinity for sodium and wherein the substrate part comprises a material comprising dietary fiber materials, polymer materials, ceramic materials, clay materials, or miscellaneous substrate materials.

A composition comprising a high-ion-affinity part and a substrate part wherein the composition is adapted for enteral delivery and wherein the high-ion-affinity part comprises a material comprising crown porphyrins and wherein the high-ion-affinity part has a high affinity for sodium and wherein the substrate part comprises a material comprising dietary fiber materials, polymer materials, ceramic materials, clay materials, or miscellaneous substrate materials.

A composition comprising a high-ion-affinity part and a substrate part wherein the composition is adapted for enteral delivery and wherein the high-ion-affinity part comprises a material comprising diestercrown ethers and wherein the high-ion-affinity part has a high affinity for sodium and wherein the substrate part comprises a material comprising dietary fiber materials, polymer materials, ceramic materials, clay materials, or miscellaneous substrate materials.

A composition comprising a high-ion-affinity part and a substrate part wherein the composition is adapted for enteral delivery and wherein the substrate part comprises a material comprising dietary fiber materials.

A composition comprising a high-ion-affinity part and a substrate part wherein the composition is adapted for enteral delivery and wherein the substrate part comprises a material comprising polymer materials.

A composition comprising a high-ion-affinity part and a substrate part wherein the composition is adapted for enteral delivery and wherein the substrate part comprises a material comprising ceramic materials.

A composition comprising a high-ion-affinity part and a substrate part wherein the composition is adapted for enteral delivery and wherein the substrate part comprises a material comprising clay materials.

A composition comprising a high-ion-affinity part and a substrate part wherein the composition is adapted for enteral delivery and wherein the substrate part comprises a material comprising miscellaneous substrate materials.

A composition comprising a high-ion-affinity part and a substrate part wherein the composition is adapted for enteral delivery and wherein the substrate part comprises a material comprising ion exchange material or kayexalate in which sodium has been substantially replaced by potassium.

A composition comprising a high-ion-affinity part and a substrate part wherein the composition is adapted for enteral delivery and wherein the substrate part comprises a material comprising dietary fiber materials and wherein the high-ion-affinity part has a high affinity for sodium.

A composition comprising a high-ion-affinity part and a substrate part wherein the composition is adapted for enteral delivery and wherein the substrate part comprises a material comprising polymer materials and wherein the high-ion-affinity part has a high affinity for sodium.

A composition comprising a high-ion-affinity part and a substrate part wherein the composition is adapted for enteral delivery and wherein the substrate part comprises a material comprising ceramic materials and wherein the high-ion-affinity part has a high affinity for sodium.

A composition comprising a high-ion-affinity part and a substrate part wherein the composition is adapted for enteral delivery and wherein the substrate part comprises a material comprising clay materials and wherein the high-ion-affinity part has a high affinity for sodium.

A composition comprising a high-ion-affinity part and a substrate part wherein the composition is adapted for enteral delivery and wherein the substrate part comprises a material comprising miscellaneous substrate materials and wherein the high-ion-affinity part has a high affinity for sodium.

A composition comprising a high-ion-affinity part and a substrate part wherein the composition is adapted for enteral delivery and wherein the substrate part comprises a material comprising ion exchange material or kayexalate in which sodium has been substantially replaced by potassium and wherein the high-ion-affinity part has a high affinity for sodium.

A composition comprising a high-ion-affinity part and a substrate part wherein the composition is adapted for enteral delivery and wherein the substrate part comprises a material comprising dietary fiber materials and wherein the high-ion-affinity part comprises a material comprising cryptands, crown ethers, azacrown ethers, thiacrown ethers, calixarenes, calixcrowns, lariat ethers, calix tubes, crown porphyrins, or diestercrown ethers.

A composition comprising a high-ion-affinity part and a substrate part wherein the composition is adapted for enteral delivery and wherein the substrate part comprises a material comprising polymer materials and wherein the high-ion-affinity part comprises a material comprising cryptands, crown ethers, azacrown ethers, thiacrown ethers, calixarenes, calixcrowns, lariat ethers, calix tubes, crown porphyrins, or diestercrown ethers.

A composition comprising a high-ion-affinity part and a substrate part wherein the composition is adapted for enteral delivery and wherein the substrate part comprises a material comprising ceramic materials and wherein the high-ion-affinity part comprises a material comprising cryptands, crown ethers, azacrown ethers, thiacrown ethers, calixarenes, calixcrowns, lariat ethers, calix tubes, crown porphyrins, or diestercrown ethers.

A composition comprising a high-ion-affinity part and a substrate part wherein the composition is adapted for enteral delivery and wherein the substrate part comprises a material comprising clay materials and wherein the high-ion-affinity part comprises a material comprising cryptands, crown ethers, azacrown ethers, thiacrown ethers, calixarenes, calixcrowns, lariat ethers, calix tubes, crown porphyrins, or diestercrown ethers.

A composition comprising a high-ion-affinity part and a substrate part wherein the composition is adapted for enteral delivery and wherein the substrate part comprises a material comprising miscellaneous substrate materials and wherein the high-ion-affinity part comprises a material comprising cryptands, crown ethers, azacrown ethers, thiacrown ethers, calixarenes, calixcrowns, lariat ethers, calix tubes, crown porphyrins, or diestercrown ethers.

A composition comprising a high-ion-affinity part and a substrate part wherein the composition is adapted for enteral delivery and wherein the substrate part comprises a material comprising ion exchange material or kayexalate in which sodium has been substantially replaced by potassium and wherein the high-ion-affinity part comprises a material comprising cryptands, crown ethers, azacrown ethers, thiacrown ethers, calixarenes, calixcrowns, lariat ethers, calix tubes, crown porphyrins, or diestercrown ethers.

A composition comprising a high-ion-affinity part and a substrate part wherein the composition is adapted for enteral delivery and wherein the substrate part comprises a material comprising dietary fiber materials and wherein the high-ion-affinity part has a high affinity for sodium and wherein the high-ion-affinity part comprises a material comprising cryptands, crown ethers, azacrown ethers, thiacrown ethers, calixarenes, calixcrowns, lariat ethers, calix tubes, crown porphyrins, or diestercrown ethers.

A composition comprising a high-ion-affinity part and a substrate part wherein the composition is adapted for enteral delivery and wherein the substrate part comprises a material comprising polymer materials and wherein the high-ion-affinity part has a high affinity for sodium and wherein the high-ion-affinity part comprises a material comprising cryptands, crown ethers, azacrown ethers, thiacrown ethers, calixarenes, calixcrowns, lariat ethers, calix tubes, crown porphyrins, or diestercrown ethers.

A composition comprising a high-ion-affinity part and a substrate part wherein the composition is adapted for enteral delivery and wherein the substrate part comprises a material comprising ceramic materials and wherein the high-ion-affinity part has a high affinity for sodium and wherein the high-ion-affinity part comprises a material comprising cryptands, crown ethers, azacrown ethers, thiacrown ethers, calixarenes, calixcrowns, lariat ethers, calix tubes, crown porphyrins, or diestercrown ethers.

A composition comprising a high-ion-affinity part and a substrate part wherein the composition is adapted for enteral delivery and wherein the substrate part comprises a material comprising clay materials and wherein the high-ion-affinity part has a high affinity for sodium and wherein the high-ion-affinity part comprises a material comprising cryptands, crown ethers, azacrown ethers, thiacrown ethers, calixarenes, calixcrowns, lariat ethers, calix tubes, crown porphyrins, or diestercrown ethers.

A composition comprising a high-ion-affinity part and a substrate part wherein the composition is adapted for enteral delivery and wherein the substrate part comprises a material comprising miscellaneous substrate materials and wherein the high-ion-affinity part has a high affinity for sodium and wherein the high-ion-affinity part comprises a material comprising cryptands, crown ethers, azacrown ethers, thiacrown ethers, calixarenes, calixcrowns, lariat ethers, calix tubes, crown porphyrins, or diestercrown ethers.

A composition comprising a high-ion-affinity part and a substrate part wherein the composition is adapted for enteral delivery and wherein the substrate part comprises a material comprising ion exchange material or kayexalate in which sodium has been substantially replaced by potassium and wherein the high-ion-affinity part has a high affinity for sodium and wherein the high-ion-affinity part comprises a material comprising cryptands, crown ethers, azacrown ethers, thiacrown ethers, calixarenes, calixcrowns, lariat ethers, calix tubes, crown porphyrins, or diestercrown ethers.

A composition comprising a high-ion-affinity part and a substrate part wherein the composition is adapted for enteral delivery and wherein the high-ion-affinity part comprises a material comprising cryptands and wherein the high-ion-affinity part has a high affinity for sodium and wherein the substrate part comprises a material comprising dietary fiber materials.

A composition comprising a high-ion-affinity part and a substrate part wherein the composition is adapted for enteral delivery and wherein the high-ion-affinity part comprises a material comprising cryptands and wherein the high-ion-affinity part has a high affinity for sodium and wherein the substrate part comprises a material comprising polymer materials.

A composition comprising a high-ion-affinity part and a substrate part wherein the composition is adapted for enteral delivery and wherein the high-ion-affinity part comprises a material comprising cryptands and wherein the high-ion-affinity part has a high affinity for sodium and wherein the substrate part comprises a material comprising ceramic materials.

A composition comprising a high-ion-affinity part and a substrate part wherein the composition is adapted for enteral delivery and wherein the high-ion-affinity part comprises a material comprising cryptands and wherein the high-ion-affinity part has a high affinity for sodium and wherein the substrate part comprises a material comprising clay materials.

A composition comprising a high-ion-affinity part and a substrate part wherein the composition is adapted for enteral delivery and wherein the high-ion-affinity part comprises a material comprising cryptands and wherein the high-ion-affinity part has a high affinity for sodium and wherein the substrate part comprises a material comprising miscellaneous substrate materials.

A composition comprising a high-ion-affinity part and a substrate part wherein the composition is adapted for enteral delivery and wherein the high-ion-affinity part comprises a material comprising cryptands and wherein the high-ion-affinity part has a high affinity for sodium and wherein the substrate part comprises a material comprising ion exchange material or kayexalate in which sodium has been substantially replaced by potassium.

A composition comprising a high-ion-affinity part and a substrate part wherein the composition is adapted for enteral delivery and wherein the high-ion-affinity part comprises a material comprising crown ethers and wherein the high-ion-affinity part has a high affinity for sodium and wherein the substrate part comprises a material comprising dietary fiber materials.

A composition comprising a high-ion-affinity part and a substrate part wherein the composition is adapted for enteral delivery and wherein the high-ion-affinity part comprises a material comprising crown ethers and wherein the high-ion-affinity part has a high affinity for sodium and wherein the substrate part comprises a material comprising polymer materials.

A composition comprising a high-ion-affinity part and a substrate part wherein the composition is adapted for enteral delivery and wherein the high-ion-affinity part comprises a material comprising crown ethers and wherein the high-ion-affinity part has a high affinity for sodium and wherein the substrate part comprises a material comprising ceramic materials.

A composition comprising a high-ion-affinity part and a substrate part wherein the composition is adapted for enteral delivery and wherein the high-ion-affinity part comprises a material comprising crown ethers and wherein the high-ion-affinity part has a high affinity for sodium and wherein the substrate part comprises a material comprising clay materials.

A composition comprising a high-ion-affinity part and a substrate part wherein the composition is adapted for enteral delivery and wherein the high-ion-affinity part comprises a material comprising crown ethers and wherein the high-ion-affinity part has a high affinity for sodium and wherein the substrate part comprises a material comprising miscellaneous substrate materials.

A composition comprising a high-ion-affinity part and a substrate part wherein the composition is adapted for enteral delivery and wherein the high-ion-affinity part comprises a material comprising crown ethers and wherein the high-ion-affinity part has a high affinity for sodium and wherein the substrate part comprises a material comprising ion exchange material or kayexalate in which sodium has been substantially replaced by potassium.

A composition comprising a high-ion-affinity part and a substrate part wherein the composition is adapted for enteral delivery and wherein the high-ion-affinity part comprises a material comprising azacrown ethers and wherein the high-ion-affinity part has a high affinity for sodium and wherein the substrate part comprises a material comprising dietary fiber materials.

A composition comprising a high-ion-affinity part and a substrate part wherein the composition is adapted for enteral delivery and wherein the high-ion-affinity part comprises a material comprising azacrown ethers and wherein the high-ion-affinity part has a high affinity for sodium and wherein the substrate part comprises a material comprising polymer materials.

A composition comprising a high-ion-affinity part and a substrate part wherein the composition is adapted for enteral delivery and wherein the high-ion-affinity part comprises a material comprising azacrown ethers and wherein the high-ion-affinity part has a high affinity for sodium and wherein the substrate part comprises a material comprising ceramic materials.

A composition comprising a high-ion-affinity part and a substrate part wherein the composition is adapted for enteral delivery and wherein the high-ion-affinity part comprises a material comprising azacrown ethers and wherein the high-ion-affinity part has a high affinity for sodium and wherein the substrate part comprises a material comprising clay materials.

A composition comprising a high-ion-affinity part and a substrate part wherein the composition is adapted for enteral delivery and wherein the high-ion-affinity part comprises a material comprising azacrown ethers and wherein the high-ion-affinity part has a high affinity for sodium and wherein the substrate part comprises a material comprising miscellaneous substrate materials.

A composition comprising a high-ion-affinity part and a substrate part wherein the composition is adapted for enteral delivery and wherein the high-ion-affinity part comprises a material comprising azacrown ethers and wherein the high-ion-affinity part has a high affinity for sodium and wherein the substrate part comprises a material comprising ion exchange material or kayexalate in which sodium has been substantially replaced by potassium.

A composition comprising a high-ion-affinity part and a substrate part wherein the composition is adapted for enteral delivery and wherein the high-ion-affinity part comprises a material comprising thiacrown ethers and wherein the high-ion-affinity part has a high affinity for sodium and wherein the substrate part comprises a material comprising dietary fiber materials.

A composition comprising a high-ion-affinity part and a substrate part wherein the composition is adapted for enteral delivery and wherein the high-ion-affinity part comprises a material comprising thiacrown ethers and wherein the high-ion-affinity part has a high affinity for sodium and wherein the substrate part comprises a material comprising polymer materials.

A composition comprising a high-ion-affinity part and a substrate part wherein the composition is adapted for enteral delivery and wherein the high-ion-affinity part comprises a material comprising thiacrown ethers and wherein the high-ion-affinity part has a high affinity for sodium and wherein the substrate part comprises a material comprising ceramic materials.

A composition comprising a high-ion-affinity part and a substrate part wherein the composition is adapted for enteral delivery and wherein the high-ion-affinity part comprises a material comprising thiacrown ethers and wherein the high-ion-affinity part has a high affinity for sodium and wherein the substrate part comprises a material comprising clay materials.

A composition comprising a high-ion-affinity part and a substrate part wherein the composition is adapted for enteral delivery and wherein the high-ion-affinity part comprises a material comprising thiacrown ethers and wherein the high-ion-affinity part has a high affinity for sodium and wherein the substrate part comprises a material comprising miscellaneous substrate materials.

A composition comprising a high-ion-affinity part and a substrate part wherein the composition is adapted for enteral delivery and wherein the high-ion-affinity part comprises a material comprising thiacrown ethers and wherein the high-ion-affinity part has a high affinity for sodium and wherein the substrate part comprises a material comprising ion exchange material or kayexalate in which sodium has been substantially replaced by potassium.

A composition comprising a high-ion-affinity part and a substrate part wherein the composition is adapted for enteral delivery and wherein the high-ion-affinity part comprises a material comprising calixarenes and wherein the high-ion-affinity part has a high affinity for sodium and wherein the substrate part comprises a material comprising dietary fiber materials.

A composition comprising a high-ion-affinity part and a substrate part wherein the composition is adapted for enteral delivery and wherein the high-ion-affinity part comprises a material comprising calixarenes and wherein the high-ion-affinity part has a high affinity for sodium and wherein the substrate part comprises a material comprising polymer materials.

A composition comprising a high-ion-affinity part and a substrate part wherein the composition is adapted for enteral delivery and wherein the high-ion-affinity part comprises a material comprising calixarenes and wherein the high-ion-affinity part has a high affinity for sodium and wherein the substrate part comprises a material comprising ceramic materials.

A composition comprising a high-ion-affinity part and a substrate part wherein the composition is adapted for enteral delivery and wherein the high-ion-affinity part comprises a material comprising calixarenes and wherein the high-ion-affinity part has a high affinity for sodium and wherein the substrate part comprises a material comprising clay materials.

A composition comprising a high-ion-affinity part and a substrate part wherein the composition is adapted for enteral delivery and wherein the high-ion-affinity part comprises a material comprising calixarenes and wherein the high-ion-affinity part has a high affinity for sodium and wherein the substrate part comprises a material comprising miscellaneous substrate materials.

A composition comprising a high-ion-affinity part and a substrate part wherein the composition is adapted for enteral delivery and wherein the high-ion-affinity part comprises a material comprising calixarenes and wherein the high-ion-affinity part has a high affinity for sodium and wherein the substrate part comprises a material comprising ion exchange material or kayexalate in which sodium has been substantially replaced by potassium.

A composition comprising a high-ion-affinity part and a substrate part wherein the composition is adapted for enteral delivery and wherein the high-ion-affinity part comprises a material comprising calixcrowns and wherein the high-ion-affinity part has a high affinity for sodium and wherein the substrate part comprises a material comprising dietary fiber materials.

A composition comprising a high-ion-affinity part and a substrate part wherein the composition is adapted for enteral delivery and wherein the high-ion-affinity part comprises a material comprising calixcrowns and wherein the high-ion-affinity part has a high affinity for sodium and wherein the substrate part comprises a material comprising polymer materials.

A composition comprising a high-ion-affinity part and a substrate part wherein the composition is adapted for enteral delivery and wherein the high-ion-affinity part comprises a material comprising calixcrowns and wherein the high-ion-affinity part has a high affinity for sodium and wherein the substrate part comprises a material comprising ceramic materials.

A composition comprising a high-ion-affinity part and a substrate part wherein the composition is adapted for enteral delivery and wherein the high-ion-affinity part comprises a material comprising calixcrowns and wherein the high-ion-affinity part has a high affinity for sodium and wherein the substrate part comprises a material comprising clay materials.

A composition comprising a high-ion-affinity part and a substrate part wherein the composition is adapted for enteral delivery and wherein the high-ion-affinity part comprises a material comprising calixcrowns and wherein the high-ion-affinity part has a high affinity for sodium and wherein the substrate part comprises a material comprising miscellaneous substrate materials.

A composition comprising a high-ion-affinity part and a substrate part wherein the composition is adapted for enteral delivery and wherein the high-ion-affinity part comprises a material comprising calixcrowns and wherein the high-ion-affinity part has a high affinity for sodium and wherein the substrate part comprises a material comprising ion exchange material or kayexalate in which sodium has been substantially replaced by potassium.

A composition comprising a high-ion-affinity part and a substrate part wherein the composition is adapted for enteral delivery and wherein the high-ion-affinity part comprises a material comprising lariat ethers and wherein the high-ion-affinity part has a high affinity for sodium and wherein the substrate part comprises a material comprising dietary fiber materials.

A composition comprising a high-ion-affinity part and a substrate part wherein the composition is adapted for enteral delivery and wherein the high-ion-affinity part comprises a material comprising lariat ethers and wherein the high-ion-affinity part has a high affinity for sodium and wherein the substrate part comprises a material comprising polymer materials.

A composition comprising a high-ion-affinity part and a substrate part wherein the composition is adapted for enteral delivery and wherein the high-ion-affinity part comprises a material comprising lariat ethers and wherein the high-ion-affinity part has a high affinity for sodium and wherein the substrate part comprises a material comprising ceramic materials.

A composition comprising a high-ion-affinity part and a substrate part wherein the composition is adapted for enteral delivery and wherein the high-ion-affinity part comprises a material comprising lariat ethers and wherein the high-ion-affinity part has a high affinity for sodium and wherein the substrate part comprises a material comprising clay materials.

A composition comprising a high-ion-affinity part and a substrate part wherein the composition is adapted for enteral delivery and wherein the high-ion-affinity part comprises a material comprising lariat ethers and wherein the high-ion-affinity part has a high affinity for sodium and wherein the substrate part comprises a material comprising miscellaneous substrate materials.

A composition comprising a high-ion-affinity part and a substrate part wherein the composition is adapted for enteral delivery and wherein the high-ion-affinity part comprises a material comprising lariat ethers and wherein the high-ion-affinity part has a high affinity for sodium and wherein the substrate part comprises a material comprising ion exchange material or kayexalate in which sodium has been substantially replaced by potassium.

A composition comprising a high-ion-affinity part and a substrate part wherein the composition is adapted for enteral delivery and wherein the high-ion-affinity part comprises a material comprising calix tubes and wherein the high-ion-affinity part has a high affinity for sodium and wherein the substrate part comprises a material comprising dietary fiber materials.

A composition comprising a high-ion-affinity part and a substrate part wherein the composition is adapted for enteral delivery and wherein the high-ion-affinity part comprises a material comprising calix tubes and wherein the high-ion-affinity part has a high affinity for sodium and wherein the substrate part comprises a material comprising polymer materials.

A composition comprising a high-ion-affinity part and a substrate part wherein the composition is adapted for enteral delivery and wherein the high-ion-affinity part comprises a material comprising calix tubes and wherein the high-ion-affinity part has a high affinity for sodium and wherein the substrate part comprises a material comprising ceramic materials.

A composition comprising a high-ion-affinity part and a substrate part wherein the composition is adapted for enteral delivery and wherein the high-ion-affinity part comprises a material comprising calix tubes and wherein the high-ion-affinity part has a high affinity for sodium and wherein the substrate part comprises a material comprising clay materials.

A composition comprising a high-ion-affinity part and a substrate part wherein the composition is adapted for enteral delivery and wherein the high-ion-affinity part comprises a material comprising calix tubes and wherein the high-ion-affinity part has a high affinity for sodium and wherein the substrate part comprises a material comprising miscellaneous substrate materials.

A composition comprising a high-ion-affinity part and a substrate part wherein the composition is adapted for enteral delivery and wherein the high-ion-affinity part comprises a material comprising calix tubes and wherein the high-ion-affinity part has a high affinity for sodium and wherein the substrate part comprises a material comprising ion exchange material or kayexalate in which sodium has been substantially replaced by potassium.

A composition comprising a high-ion-affinity part and a substrate part wherein the composition is adapted for enteral delivery and wherein the high-ion-affinity part comprises a material comprising crown porphyrins and wherein the high-ion-affinity part has a high affinity for sodium and wherein the substrate part comprises a material comprising dietary fiber materials.

A composition comprising a high-ion-affinity part and a substrate part wherein the composition is adapted for enteral delivery and wherein the high-ion-affinity part comprises a material comprising crown porphyrins and wherein the high-ion-affinity part has a high affinity for sodium and wherein the substrate part comprises a material comprising polymer materials.

A composition comprising a high-ion-affinity part and a substrate part wherein the composition is adapted for enteral delivery and wherein the high-ion-affinity part comprises a material comprising crown porphyrins and wherein the high-ion-affinity part has a high affinity for sodium and wherein the substrate part comprises a material comprising ceramic materials.

A composition comprising a high-ion-affinity part and a substrate part wherein the composition is adapted for enteral delivery and wherein the high-ion-affinity part comprises a material comprising crown porphyrins and wherein the high-ion-affinity part has a high affinity for sodium and wherein the substrate part comprises a material comprising clay materials.

A composition comprising a high-ion-affinity part and a substrate part wherein the composition is adapted for enteral delivery and wherein the high-ion-affinity part comprises a material comprising crown porphyrins and wherein the high-ion-affinity part has a high affinity for sodium and wherein the substrate part comprises a material comprising miscellaneous substrate materials.

A composition comprising a high-ion-affinity part and a substrate part wherein the composition is adapted for enteral delivery and wherein the high-ion-affinity part comprises a material comprising crown porphyrins and wherein the high-ion-affinity part has a high affinity for sodium and wherein the substrate part comprises a material comprising ion exchange material or kayexalate in which sodium has been substantially replaced by potassium.

A composition comprising a high-ion-affinity part and a substrate part wherein the composition is adapted for enteral delivery and wherein the high-ion-affinity part comprises a material comprising diestercrown ethers and wherein the high-ion-affinity part has a high affinity for sodium and wherein the substrate part comprises a material comprising dietary fiber materials.

A composition comprising a high-ion-affinity part and a substrate part wherein the composition is adapted for enteral delivery and wherein the high-ion-affinity part comprises a material comprising diestercrown ethers and wherein the high-ion-affinity part has a high affinity for sodium and wherein the substrate part comprises a material comprising polymer materials.

A composition comprising a high-ion-affinity part and a substrate part wherein the composition is adapted for enteral delivery and wherein the high-ion-affinity part comprises a material comprising diestercrown ethers and wherein the high-ion-affinity part has a high affinity for sodium and wherein the substrate part comprises a material comprising ceramic materials.

A composition comprising a high-ion-affinity part and a substrate part wherein the composition is adapted for enteral delivery and wherein the high-ion-affinity part comprises a material comprising diestercrown ethers and wherein the high-ion-affinity part has a high affinity for sodium and wherein the substrate part comprises a material comprising clay materials.

A composition comprising a high-ion-affinity part and a substrate part wherein the composition is adapted for enteral delivery and wherein the high-ion-affinity part comprises a material comprising diestercrown ethers and wherein the high-ion-affinity part has a high affinity for sodium and wherein the substrate part comprises a material comprising miscellaneous substrate materials.

A composition comprising a high-ion-affinity part and a substrate part wherein the composition is adapted for enteral delivery and wherein the high-ion-affinity part comprises a material comprising diestercrown ethers and wherein the high-ion-affinity part has a high affinity for sodium and wherein the substrate part comprises a material comprising ion exchange material or kayexalate in which sodium has been substantially replaced by potassium.

A composition comprising a high-ion-affinity part and a substrate part wherein the composition is adapted for enteral delivery and wherein the substrate part comprises a material comprising dietary fiber materials and wherein the high-ion-affinity part comprises a material comprising [2.2.2]-crypt.

A composition comprising a high-ion-affinity part and a substrate part wherein the composition is adapted for enteral delivery and wherein the substrate part comprises a material comprising polymer materials and wherein the high-ion-affinity part comprises a material comprising [2.2.2]-crypt.

A composition comprising a high-ion-affinity part and a substrate part wherein the composition is adapted for enteral delivery and wherein the substrate part comprises a material comprising ceramic materials and wherein the high-ion-affinity part comprises a material comprising [2.2.2]-crypt.

A composition comprising a high-ion-affinity part and a substrate part wherein the composition is adapted for enteral delivery and wherein the substrate part comprises a material comprising clay materials and wherein the high-ion-affinity part comprises a material comprising [2.2.2]-crypt.

A composition comprising a high-ion-affinity part and a substrate part wherein the composition is adapted for enteral delivery and wherein the substrate part comprises a material comprising miscellaneous substrate materials and wherein the high-ion-affinity part comprises a material comprising [2.2.2]-crypt.

A composition comprising a high-ion-affinity part and a substrate part wherein the composition is adapted for enteral delivery and wherein the substrate part comprises a material comprising ion exchange material or kayexalate in which sodium has been substantially replaced by potassium and wherein the high-ion-affinity part comprises a material comprising [2.2.2]-crypt.

A composition comprising a high-ion-affinity part and a substrate part wherein the composition is adapted for enteral delivery and wherein the high-ion-affinity part comprises a material comprising cryptands and wherein the substrate part comprises psyllium fibers.

A composition comprising a high-ion-affinity part and a substrate part wherein the composition is adapted for enteral delivery and wherein the high-ion-affinity part comprises a material comprising crown ethers and wherein the substrate part comprises psyllium fibers.

A composition comprising a high-ion-affinity part and a substrate part wherein the composition is adapted for enteral delivery and wherein the high-ion-affinity part comprises a material comprising azacrown ethers and wherein the substrate part comprises psyllium fibers.

A composition comprising a high-ion-affinity part and a substrate part wherein the composition is adapted for enteral delivery and wherein the high-ion-affinity part comprises a material comprising thiacrown ethers and wherein the substrate part comprises psyllium fibers.

A composition comprising a high-ion-affinity part and a substrate part wherein the composition is adapted for enteral delivery and wherein the high-ion-affinity part comprises a material comprising calixarenes and wherein the substrate part comprises psyllium fibers.

A composition comprising a high-ion-affinity part and a substrate part wherein the composition is adapted for enteral delivery and wherein the high-ion-affinity part comprises a material comprising calixcrowns and wherein the substrate part comprises psyllium fibers.

A composition comprising a high-ion-affinity part and a substrate part wherein the composition is adapted for enteral delivery and wherein the high-ion-affinity part comprises a material comprising lariat ethers and wherein the substrate part comprises psyllium fibers.

A composition comprising a high-ion-affinity part and a substrate part wherein the composition is adapted for enteral delivery and wherein the high-ion-affinity part comprises a material comprising calix tubes and wherein the substrate part comprises psyllium fibers.

A composition comprising a high-ion-affinity part and a substrate part wherein the composition is adapted for enteral delivery and wherein the high-ion-affinity part comprises a material comprising crown porphyrins and wherein the substrate part comprises psyllium fibers.

A composition comprising a high-ion-affinity part and a substrate part wherein the composition is adapted for enteral delivery and wherein the high-ion-affinity part comprises a material comprising diestercrown ethers and wherein the substrate part comprises psyllium fibers.

A composition comprising a high-ion-affinity part and a substrate part wherein the composition is adapted for enteral delivery and wherein the high-ion-affinity part comprises a material comprising cryptands and wherein the high-ion-affinity part has a high ion affinity for sodium and wherein the substrate part comprises psyllium fibers.

A composition comprising a high-ion-affinity part and a substrate part wherein the composition is adapted for enteral delivery and wherein the high-ion-affinity part comprises a material comprising crown ethers and wherein the high-ion-affinity part has a high ion affinity for sodium and wherein the substrate part comprises psyllium fibers.

A composition comprising a high-ion-affinity part and a substrate part wherein the composition is adapted for enteral delivery and wherein the high-ion-affinity part comprises a material comprising azacrown ethers and wherein the high-ion-affinity part has a high ion affinity for sodium and wherein the substrate part comprises psyllium fibers.

A composition comprising a high-ion-affinity part and a substrate part wherein the composition is adapted for enteral delivery and wherein the high-ion-affinity part comprises a material comprising thiacrown ethers and wherein the high-ion-affinity part has a high ion affinity for sodium and wherein the substrate part comprises psyllium fibers.

A composition comprising a high-ion-affinity part and a substrate part wherein the composition is adapted for enteral delivery and wherein the high-ion-affinity part comprises a material comprising calixarenes and wherein the high-ion-affinity part has a high ion affinity for sodium and wherein the substrate part comprises psyllium fibers.

A composition comprising a high-ion-affinity part and a substrate part wherein the composition is adapted for enteral delivery and wherein the high-ion-affinity part comprises a material comprising calixcrowns and wherein the high-ion-affinity part has a high ion affinity for sodium and wherein the substrate part comprises psyllium fibers.

A composition comprising a high-ion-affinity part and a substrate part wherein the composition is adapted for enteral delivery and wherein the high-ion-affinity part comprises a material comprising lariat ethers and wherein the high-ion-affinity part has a high ion affinity for sodium and wherein the substrate part comprises psyllium fibers.

A composition comprising a high-ion-affinity part and a substrate part wherein the composition is adapted for enteral delivery and wherein the high-ion-affinity part comprises a material comprising calix tubes and wherein the high-ion-affinity part has a high ion affinity for sodium and wherein the substrate part comprises psyllium fibers.

A composition comprising a high-ion-affinity part and a substrate part wherein the composition is adapted for enteral delivery and wherein the high-ion-affinity part comprises a material comprising crown porphyrins and wherein the high-ion-affinity part has a high ion affinity for sodium and wherein the substrate part comprises psyllium fibers.

A composition comprising a high-ion-affinity part and a substrate part wherein the composition is adapted for enteral delivery and wherein the high-ion-affinity part comprises a material comprising diestercrown ethers and wherein the high-ion-affinity part has a high ion affinity for sodium and wherein the substrate part comprises psyllium fibers.

A composition comprising a high-ion-affinity part and a substrate part wherein the composition is adapted for enteral delivery and wherein the high-ion-affinity part comprises a material comprising cryptands and wherein the substrate part comprises kayexalate in which sodium has been substantially replaced by potassium.

A composition comprising a high-ion-affinity part and a substrate part wherein the composition is adapted for enteral delivery and wherein the high-ion-affinity part comprises a material comprising crown ethers and wherein the substrate part comprises kayexalate in which sodium has been substantially replaced by potassium.

A composition comprising a high-ion-affinity part and a substrate part wherein the composition is adapted for enteral delivery and wherein the high-ion-affinity part comprises a material comprising azacrown ethers and wherein the substrate part comprises kayexalate in which sodium has been substantially replaced by potassium.

A composition comprising a high-ion-affinity part and a substrate part wherein the composition is adapted for enteral delivery and wherein the high-ion-affinity part comprises a material comprising thiacrown ethers and wherein the substrate part comprises kayexalate in which sodium has been substantially replaced by potassium.

A composition comprising a high-ion-affinity part and a substrate part wherein the composition is adapted for enteral delivery and wherein the high-ion-affinity part comprises a material comprising calixarenes and wherein the substrate part comprises kayexalate in which sodium has been substantially replaced by potassium.

A composition comprising a high-ion-affinity part and a substrate part wherein the composition is adapted for enteral delivery and wherein the high-ion-affinity part comprises a material comprising calixcrowns and wherein the substrate part comprises kayexalate in which sodium has been substantially replaced by potassium.

A composition comprising a high-ion-affinity part and a substrate part wherein the composition is adapted for enteral delivery and wherein the high-ion-affinity part comprises a material comprising lariat ethers and wherein the substrate part comprises kayexalate in which sodium has been substantially replaced by potassium.

A composition comprising a high-ion-affinity part and a substrate part wherein the composition is adapted for enteral delivery and wherein the high-ion-affinity part comprises a material comprising calix tubes and wherein the substrate part comprises kayexalate in which sodium has been substantially replaced by potassium.

A composition comprising a high-ion-affinity part and a substrate part wherein the composition is adapted for enteral delivery and wherein the high-ion-affinity part comprises a material comprising crown porphyrins and wherein the substrate part comprises kayexalate in which sodium has been substantially replaced by potassium.

A composition comprising a high-ion-affinity part and a substrate part wherein the composition is adapted for enteral delivery and wherein the high-ion-affinity part comprises a material comprising diestercrown ethers and wherein the substrate part comprises kayexalate in which sodium has been substantially replaced by potassium.

A composition comprising a high-ion-affinity part and a substrate part wherein the composition is adapted for enteral delivery and wherein the high-ion-affinity part comprises a material comprising cryptands and wherein the high-ion-affinity part has a high ion affinity for sodium and wherein the substrate part comprises kayexalate in which sodium has been substantially replaced by potassium.

A composition comprising a high-ion-affinity part and a substrate part wherein the composition is adapted for enteral delivery and wherein the high-ion-affinity part comprises a material comprising crown ethers and wherein the high-ion-affinity part has a high ion affinity for sodium and wherein the substrate part comprises kayexalate in which sodium has been substantially replaced by potassium.

A composition comprising a high-ion-affinity part and a substrate part wherein the composition is adapted for enteral delivery and wherein the high-ion-affinity part comprises a material comprising azacrown ethers and wherein the high-ion-affinity part has a high ion affinity for sodium and wherein the substrate part comprises kayexalate in which sodium has been substantially replaced by potassium.

A composition comprising a high-ion-affinity part and a substrate part wherein the composition is adapted for enteral delivery and wherein the high-ion-affinity part comprises a material comprising thiacrown ethers and wherein the high-ion-affinity part has a high ion affinity for sodium and wherein the substrate part comprises kayexalate in which sodium has been substantially replaced by potassium.

A composition comprising a high-ion-affinity part and a substrate part wherein the composition is adapted for enteral delivery and wherein the high-ion-affinity part comprises a material comprising calixarenes and wherein the high-ion-affinity part has a high ion affinity for sodium and wherein the substrate part comprises kayexalate in which sodium has been substantially replaced by potassium.

A composition comprising a high-ion-affinity part and a substrate part wherein the composition is adapted for enteral delivery and wherein the high-ion-affinity part comprises a material comprising calixcrowns and wherein the high-ion-affinity part has a high ion affinity for sodium and wherein the substrate part comprises kayexalate in which sodium has been substantially replaced by potassium.

A composition comprising a high-ion-affinity part and a substrate part wherein the composition is adapted for enteral delivery and wherein the high-ion-affinity part comprises a material comprising lariat ethers and wherein the high-ion-affinity part has a high ion affinity for sodium and wherein the substrate part comprises kayexalate in which sodium has been substantially replaced by potassium.

A composition comprising a high-ion-affinity part and a substrate part wherein the composition is adapted for enteral delivery and wherein the high-ion-affinity part comprises a material comprising calix tubes and wherein the high-ion-affinity part has a high ion affinity for sodium and wherein the substrate part comprises kayexalate in which sodium has been substantially replaced by potassium.

A composition comprising a high-ion-affinity part and a substrate part wherein the composition is adapted for enteral delivery and wherein the high-ion-affinity part comprises a material comprising crown porphyrins and wherein the high-ion-affinity part has a high ion affinity for sodium and wherein the substrate part comprises kayexalate in which sodium has been substantially replaced by potassium.

A composition comprising a high-ion-affinity part and a substrate part wherein the composition is adapted for enteral delivery and wherein the high-ion-affinity part comprises a material comprising diestercrown ethers and wherein the high-ion-affinity part has a high ion affinity for sodium and wherein the substrate part comprises kayexalate in which sodium has been substantially replaced by potassium.

A composition comprising a high-ion-affinity part and a substrate part wherein the composition is adapted for enteral delivery and wherein the high-ion-affinity part comprises a material comprising cryptands and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the high-ion-affinity part comprises a material comprising crown ethers and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the high-ion-affinity part comprises a material comprising cryptands and wherein the high-ion-affinity part has a high affinity for sodium and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the high-ion-affinity part comprises a material comprising crown ethers and wherein the high-ion-affinity part has a high affinity for sodium and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the high-ion-affinity part comprises a material comprising cryptands and wherein the substrate part comprises a material comprising dietary fiber materials, polymer materials, ceramic materials, clay materials, or miscellaneous substrate materials and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the high-ion-affinity part comprises a material comprising crown ethers and wherein the substrate part comprises a material comprising dietary fiber materials, polymer materials, ceramic materials, clay materials, or miscellaneous substrate materials and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the high-ion-affinity part comprises a material comprising cryptands and wherein the high-ion-affinity part has a high affinity for sodium and wherein the substrate part comprises a material comprising dietary fiber materials, polymer materials, ceramic materials, clay materials, or miscellaneous substrate materials and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the high-ion-affinity part comprises a material comprising crown ethers and wherein the high-ion-affinity part has a high affinity for sodium and wherein the substrate part comprises a material comprising dietary fiber materials, polymer materials, ceramic materials, clay materials, or miscellaneous substrate materials and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the substrate part comprises a material comprising dietary fiber materials and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the substrate part comprises a material comprising polymer materials and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the substrate part comprises a material comprising ceramic materials and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the substrate part comprises a material comprising clay materials and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the substrate part comprises a material comprising miscellaneous substrate materials and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the substrate part comprises a material comprising ion exchange material or kayexalate in which sodium has been substantially replaced by potassium and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the substrate part comprises a material comprising dietary fiber materials and wherein the high-ion-affinity part has a high affinity for sodium and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the substrate part comprises a material comprising polymer materials and wherein the high-ion-affinity part has a high affinity for sodium and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the substrate part comprises a material comprising ceramic materials and wherein the high-ion-affinity part has a high affinity for sodium and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the substrate part comprises a material comprising clay materials and wherein the high-ion-affinity part has a high affinity for sodium and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the substrate part comprises a material comprising miscellaneous substrate materials and wherein the high-ion-affinity part has a high affinity for sodium and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the substrate part comprises a material comprising ion exchange material or kayexalate in which sodium has been substantially replaced by potassium and wherein the high-ion-affinity part has a high affinity for sodium and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the high-ion-affinity part comprises a material comprising cryptands and wherein the high-ion-affinity part has a high affinity for sodium and wherein the substrate part comprises a material comprising dietary fiber materials and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the high-ion-affinity part comprises a material comprising cryptands and wherein the high-ion-affinity part has a high affinity for sodium and wherein the substrate part comprises a material comprising polymer materials and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the high-ion-affinity part comprises a material comprising cryptands and wherein the high-ion-affinity part has a high affinity for sodium and wherein the substrate part comprises a material comprising ceramic materials and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the high-ion-affinity part comprises a material comprising cryptands and wherein the high-ion-affinity part has a high affinity for sodium and wherein the substrate part comprises a material comprising clay materials and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the high-ion-affinity part comprises a material comprising cryptands and wherein the high-ion-affinity part has a high affinity for sodium and wherein the substrate part comprises a material comprising miscellaneous substrate materials and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the high-ion-affinity part comprises a material comprising cryptands and wherein the high-ion-affinity part has a high affinity for sodium and wherein the substrate part comprises a material comprising ion exchange material or kayexalate in which sodium has been substantially replaced by potassium and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the high-ion-affinity part comprises a material comprising crown ethers and wherein the high-ion-affinity part has a high affinity for sodium and wherein the substrate part comprises a material comprising dietary fiber materials and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the high-ion-affinity part comprises a material comprising crown ethers and wherein the high-ion-affinity part has a high affinity for sodium and wherein the substrate part comprises a material comprising polymer materials and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the high-ion-affinity part comprises a material comprising crown ethers and wherein the high-ion-affinity part has a high affinity for sodium and wherein the substrate part comprises a material comprising ceramic materials and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the high-ion-affinity part comprises a material comprising crown ethers and wherein the high-ion-affinity part has a high affinity for sodium and wherein the substrate part comprises a material comprising clay materials and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the high-ion-affinity part comprises a material comprising crown ethers and wherein the high-ion-affinity part has a high affinity for sodium and wherein the substrate part comprises a material comprising miscellaneous substrate materials and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the high-ion-affinity part comprises a material comprising crown ethers and wherein the high-ion-affinity part has a high affinity for sodium and wherein the substrate part comprises a material comprising ion exchange material or kayexalate in which sodium has been substantially replaced by potassium and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the substrate part comprises a material comprising dietary fiber materials and wherein the high-ion-affinity part comprises a material comprising [2,2,2]-crypt and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the substrate part comprises a material comprising polymer materials and wherein the high-ion-affinity part comprises a material comprising [2,2,2]-crypt and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the substrate part comprises a material comprising ceramic materials and wherein the high-ion-affinity part comprises a material comprising [2,2,2]-crypt and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the substrate part comprises a material comprising clay materials and wherein the high-ion-affinity part comprises a material comprising [2,2,2]-crypt and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the substrate part comprises a material comprising miscellaneous substrate materials and wherein the high-ion-affinity part comprises a material comprising [2,2,2]-crypt and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the substrate part comprises a material comprising ion exchange material or kayexalate in which sodium has been substantially replaced by potassium and wherein the high-ion-affinity part comprises a material comprising [2,2,2]-crypt and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the high-ion-affinity part comprises a material comprising cryptands and wherein the substrate part comprises psyllium fibers and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the high-ion-affinity part comprises a material comprising crown ethers and wherein the substrate part comprises psyllium fibers and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the high-ion-affinity part comprises a material comprising cryptands and wherein the high-ion-affinity part has a high ion affinity for sodium and wherein the substrate part comprises psyllium fibers and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the high-ion-affinity part comprises a material comprising crown ethers and wherein the high-ion-affinity part has a high ion affinity for sodium and wherein the substrate part comprises psyllium fibers and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the high-ion-affinity part comprises a material comprising cryptands and wherein the substrate part comprises kayexalate in which sodium has been substantially replaced by potassium and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the high-ion-affinity part comprises a material comprising crown ethers and wherein the substrate part comprises kayexalate in which sodium has been substantially replaced by potassium and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the high-ion-affinity part comprises a material comprising cryptands and wherein the high-ion-affinity part has a high ion affinity for sodium and wherein the substrate part comprises kayexalate in which sodium has been substantially replaced by potassium and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the high-ion-affinity part comprises a material comprising crown ethers and wherein the high-ion-affinity part has a high ion affinity for sodium and wherein the substrate part comprises kayexalate in which sodium has been substantially replaced by potassium and wherein the composition further comprises one or more hypertension drugs such as angiotensin-converting enzyme (ace) inhibitors; angiotensin ii receptor blockers; beta blockers; calcium channel blockers; renin inhibitors; or thiazide diuretics.

A composition comprising a high-ion-affinity part and a substrate part wherein the composition is adapted for enteral delivery and wherein the high-ion-affinity part comprises a material comprising cryptands and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the high-ion-affinity part comprises a material comprising crown ethers and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the high-ion-affinity part comprises a material comprising cryptands and wherein the high-ion-affinity part has a high affinity for sodium and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the high-ion-affinity part comprises a material comprising crown ethers and wherein the high-ion-affinity part has a high affinity for sodium and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the high-ion-affinity part comprises a material comprising cryptands and wherein the substrate part comprises a material comprising dietary fiber materials, polymer materials, ceramic materials, clay materials, or miscellaneous substrate materials and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the high-ion-affinity part comprises a material comprising crown ethers and wherein the substrate part comprises a material comprising dietary fiber materials, polymer materials, ceramic materials, clay materials, or miscellaneous substrate materials and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the high-ion-affinity part comprises a material comprising cryptands and wherein the high-ion-affinity part has a high affinity for sodium and wherein the substrate part comprises a material comprising dietary fiber materials, polymer materials, ceramic materials, clay materials, or miscellaneous substrate materials and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the high-ion-affinity part comprises a material comprising crown ethers and wherein the high-ion-affinity part has a high affinity for sodium and wherein the substrate part comprises a material comprising dietary fiber materials, polymer materials, ceramic materials, clay materials, or miscellaneous substrate materials and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the substrate part comprises a material comprising dietary fiber materials and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the substrate part comprises a material comprising polymer materials and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the substrate part comprises a material comprising ceramic materials and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the substrate part comprises a material comprising clay materials and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the substrate part comprises a material comprising miscellaneous substrate materials and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the substrate part comprises a material comprising ion exchange material or kayexalate in which sodium has been substantially replaced by potassium and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the substrate part comprises a material comprising dietary fiber materials and wherein the high-ion-affinity part has a high affinity for sodium and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the substrate part comprises a material comprising polymer materials and wherein the high-ion-affinity part has a high affinity for sodium and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the substrate part comprises a material comprising ceramic materials and wherein the high-ion-affinity part has a high affinity for sodium and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the substrate part comprises a material comprising clay materials and wherein the high-ion-affinity part has a high affinity for sodium and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the substrate part comprises a material comprising miscellaneous substrate materials and wherein the high-ion-affinity part has a high affinity for sodium and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the substrate part comprises a material comprising ion exchange material or kayexalate in which sodium has been substantially replaced by potassium and wherein the high-ion-affinity part has a high affinity for sodium and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the high-ion-affinity part comprises a material comprising cryptands and wherein the high-ion-affinity part has a high affinity for sodium and wherein the substrate part comprises a material comprising dietary fiber materials and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the high-ion-affinity part comprises a material comprising cryptands and wherein the high-ion-affinity part has a high affinity for sodium and wherein the substrate part comprises a material comprising polymer materials and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the high-ion-affinity part comprises a material comprising cryptands and wherein the high-ion-affinity part has a high affinity for sodium and wherein the substrate part comprises a material comprising ceramic materials and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the high-ion-affinity part comprises a material comprising cryptands and wherein the high-ion-affinity part has a high affinity for sodium and wherein the substrate part comprises a material comprising clay materials and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the high-ion-affinity part comprises a material comprising cryptands and wherein the high-ion-affinity part has a high affinity for sodium and wherein the substrate part comprises a material comprising miscellaneous substrate materials and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the high-ion-affinity part comprises a material comprising cryptands and wherein the high-ion-affinity part has a high affinity for sodium and wherein the substrate part comprises a material comprising ion exchange material or kayexalate in which sodium has been substantially replaced by potassium and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the high-ion-affinity part comprises a material comprising crown ethers and wherein the high-ion-affinity part has a high affinity for sodium and wherein the substrate part comprises a material comprising dietary fiber materials and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the high-ion-affinity part comprises a material comprising crown ethers and wherein the high-ion-affinity part has a high affinity for sodium and wherein the substrate part comprises a material comprising polymer materials and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the high-ion-affinity part comprises a material comprising crown ethers and wherein the high-ion-affinity part has a high affinity for sodium and wherein the substrate part comprises a material comprising ceramic materials and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the high-ion-affinity part comprises a material comprising crown ethers and wherein the high-ion-affinity part has a high affinity for sodium and wherein the substrate part comprises a material comprising clay materials and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the high-ion-affinity part comprises a material comprising crown ethers and wherein the high-ion-affinity part has a high affinity for sodium and wherein the substrate part comprises a material comprising miscellaneous substrate materials and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the high-ion-affinity part comprises a material comprising crown ethers and wherein the high-ion-affinity part has a high affinity for sodium and wherein the substrate part comprises a material comprising ion exchange material or kayexalate in which sodium has been substantially replaced by potassium and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the substrate part comprises a material comprising dietary fiber materials and wherein the high-ion-affinity part comprises a material comprising [2,2,2]-crypt and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the substrate part comprises a material comprising polymer materials and wherein the high-ion-affinity part comprises a material comprising [2,2,2]-crypt and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the substrate part comprises a material comprising ceramic materials and wherein the high-ion-affinity part comprises a material comprising [2,2,2]-crypt and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the substrate part comprises a material comprising clay materials and wherein the high-ion-affinity part comprises a material comprising [2,2,2]-crypt and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the substrate part comprises a material comprising miscellaneous substrate materials and wherein the high-ion-affinity part comprises a material comprising [2,2,2]-crypt and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the substrate part comprises a material comprising ion exchange material or kayexalate in which sodium has been substantially replaced by potassium and wherein the high-ion-affinity part comprises a material comprising [2,2,2]-crypt and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the high-ion-affinity part comprises a material comprising cryptands and wherein the substrate part comprises psyllium fibers and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the high-ion-affinity part comprises a material comprising crown ethers and wherein the substrate part comprises psyllium fibers and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the high-ion-affinity part comprises a material comprising cryptands and wherein the high-ion-affinity part has a high ion affinity for sodium and wherein the substrate part comprises psyllium fibers and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the high-ion-affinity part comprises a material comprising crown ethers and wherein the high-ion-affinity part has a high ion affinity for sodium and wherein the substrate part comprises psyllium fibers and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the high-ion-affinity part comprises a material comprising cryptands and wherein the substrate part comprises kayexalate in which sodium has been substantially replaced by potassium and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the high-ion-affinity part comprises a material comprising crown ethers and wherein the substrate part comprises kayexalate in which sodium has been substantially replaced by potassium and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the high-ion-affinity part comprises a material comprising cryptands and wherein the high-ion-affinity part has a high ion affinity for sodium and wherein the substrate part comprises kayexalate in which sodium has been substantially replaced by potassium and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

A composition comprising a high-ion-affinity part and a substrate part wherein the composition is adapted for enteral delivery and wherein the high-ion-affinity part comprises a material comprising crown ethers and wherein the high-ion-affinity part has a high ion affinity for sodium and wherein the substrate part comprises kayexalate in which sodium has been substantially replaced by potassium and wherein the composition further comprises one or more cholesterol or triglyceride lowering drugs such as lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.

While particular embodiments of the present invention have been shown and described, it will be obvious to those skilled in the art that changes and modifications can be made without departing from the embodiments of this invention in its broader aspects and, therefore, the appended claims are to encompass within their scope all such changes and modifications as fall within the true, intended, explained, disclose, and understood scope and spirit of this invention's multitudinous embodiments and alternative descriptions.

Additionally, various embodiments have been described above. For convenience's sake, combinations of aspects composing invention embodiments have been listed in such a way that one of ordinary skill in the art may read them exclusive of each other when they are not necessarily intended to be exclusive. But a recitation of an aspect for one embodiment is meant to disclose its use in all embodiments in which that aspect can be incorporated without undue experimentation. In like manner, a recitation of an aspect as composing part of an embodiment is a tacit recognition that a supplementary embodiment exists that specifically excludes that aspect. All patents, test procedures, and other documents cited in this specification are fully incorporated by reference to the extent that this material is consistent with this specification and for all jurisdictions in which such incorporation is permitted.

Moreover, some embodiments recite ranges. When this is done, it is meant to disclose the ranges as a range, and to disclose each and every point within the range, including end points. For those embodiments that disclose a specific value or condition for an aspect, supplementary embodiments exist that are otherwise identical, but that specifically exclude the value or the conditions for the aspect.

Finally, headings are for the convenience of the reader and do not alter the meaning or content of the disclosure or the scope of the claims.

1. A composition comprising a high-ion-affinity part and a substrate part wherein the composition is adapted for enteral delivery.

2. The composition of claim 1 wherein the high-ion-affinity part has a high affinity for ions present in the intestinal tract of an animal.

3. The composition of claim 2 wherein the ion present in the intestinal tract of an animal is sodium.

4. The composition of claim 1 wherein the high-ion-affinity part comprises a material comprising cryptands, crown ethers, azacrown ethers, thiacrown ethers, calixarenes, calixcrowns, lariat ethers, calix tubes, crown porphyrins, or diestercrown ethers.

5. The composition of claim 4 wherein the high-ion-affinity part comprises a material comprising cryptands.

6. The composition of claim 5 wherein the substrate part comprises a material comprising dietary fiber materials, polymer materials, ceramic materials, clay materials, or miscellaneous substrate materials.

7. The composition of claim 6 wherein the substrate part comprises a material comprising dietary fiber materials.

8. The composition of claim 6 wherein the substrate part comprises a material comprising ion exchange material or kayexalate.

9. The composition of claim 4 wherein the substrate part comprises a material comprising dietary fiber materials, polymer materials, ceramic materials, clay materials, or miscellaneous substrate materials.

10. The composition of claim 9 wherein the substrate part comprises a material comprising dietary fiber materials.

11. The composition of claim 9 wherein the substrate part comprises a material comprising ion exchange material or kayexalate.

12. The composition of claim 3 wherein the high-ion-affinity part comprises a material comprising cryptands, crown ethers, azacrown ethers, thiacrown ethers, calixarenes, calixcrowns, lariat ethers, calix tubes, crown porphyrins, or diestercrown ethers.

13. The composition of claim 12 wherein the high-ion-affinity part comprises a material comprising cryptands.

14. The composition of claim 13 wherein the substrate part comprises a material comprising dietary fiber materials, polymer materials, ceramic materials, clay materials, or miscellaneous substrate materials.

15. The composition of claim 14 wherein the substrate part comprises a material comprising dietary fiber materials.

16. The composition of claim 14 wherein the substrate part comprises a material comprising ion exchange material or kayexalate.

17. The composition of claim 12 wherein the substrate part comprises a material comprising dietary fiber materials.

18. The composition of claim 12 wherein the substrate part comprises a material comprising ion exchange material or kayexalate.

19. The composition of claim 1 wherein the composition further comprises one or more drugs selected from any one or any combination of hypertension drugs, high-cholesterol-treating drugs, or high-triglyceride-treating drugs.

20. The composition of claim 19 wherein the composition further comprises one or more drugs selected from any one or any combination of acebutolol; acetazolamide; aliskiren; amiloride and hydrochlorothiazide; amiloride hydrochloride; amlodipine; amlodipine and benazepril; atenolol; atenolol and chlorthalidone; benazepril; benazepril and hydrochlorothiazide; betaxolol; bisoprolol; bisoprolol and hydrochlorothiazide; bumetanide; candesartan; captopril; captopril and hydrochlorothiazide; carteolol; carvedilol; chlorthalidone; clonidine; diltiazem; doxazosin; enalapril; enalapril and hydrochlorothiazide; esidrix; ethacrynic acid; felodipine; felodipine and enalapril; fosinopril; furosemide; guanabenz; guanfacine; hydralazine; hydralazine and hydrochlorothiazide; hydrochlorothiazide; indapamide; irbesartan; isradipine; labetalol; lisinopril; lisinopril and hydrochlorothiazide; losartan; losartan and hydrochlorothiazide; methyldopa; methyldopa and hydrochlorothiazide; metolazone; metoprolol; metoprolol and hydrochlorothiazide; microzide; moexipril; nadolol; nadolol and bendroflumethiazide; nicardipine; nifedipine; nisoldipine; olmesartan; penbutolol; prazosin; propranolol; propranolol and hydrochlorothiazide; quinapril; ramipril; reserpine; spironolactone; spironolactone and hydrochlorothiazide; telmisartan; terazosin; timolol; torsemide; trandolapril; triamterene; triamterene and hydrochlorothiazide; valsartan; verapamil; verapamil (extended release) and trandolapril; lovastatin; simvastatin; atorvastatin; fluvastatin; pravastatin; rosuvastatin; fenofibrate; ezetimibe; niacin; fenofibric acid; bile acid sequestrants; omega-3-acid ethyl esters; cholestyramine resin; colesevelam hydrochloride; red yeast rice; gemfibrozil; evening primrose oil; cerivastatin; clofibrate; dextrothyroxine sodium; or pitavastatin.
